(19) United States
(12) Patent Application Publication
(10) Pub. No.: US 2023/0096443 A1
(43) Pub. Date: Mar. 30, 2023

(54) NOVEL NICOTINAMIDE DI-NUCLEOTIDE DERIVATIVES AND USE THEREOF

(71) Applicant: NUVAMID SA, Epalinges (CH)

(72) Inventors: Guillaume BERMOND, Epalinges (CH); Laurent GARÇON, Epalinges (CH)

(73) Assignee: NUVAMID SA, Epalinges (CH)

(21) Appl. No.: 17/785,631

(22) PCT Filed: Dec. 18, 2020
(86) PCT No.: PCT/EP2020/087330

§ 371 Date: Jun. 15, 2022

(30) Foreign Application Priority Data
Dec. 20, 2019 (EP) 19218817.5

(51) Int. Cl. C07D 405/14; C07H 21/02

(52) U.S. Cl. CPC C07D 405/14; C07H 21/02

(57) ABSTRACT

A compound of formula I or pharmaceutically acceptable salts and/or solvates thereof:

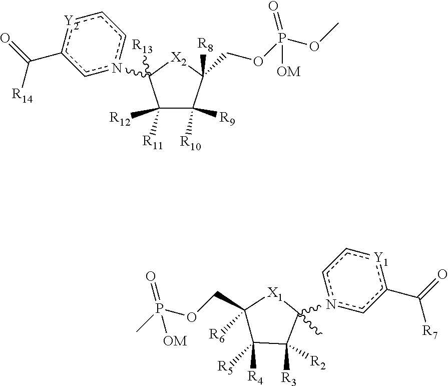

Also, compositions including at least one compound of formula I, such a pharmaceutical composition, a food composition, and a cosmetic composition. Further, a method for preparing compounds of formula I and their use as therapeutic compounds for use in the treatment of pain, antineoplastic-induced cardiotoxicity or sickle cell disease.

### FIELD OF INVENTION

The present invention relates to novel nicotinamide di-nucleotide derivatives including their pharmaceutically acceptable salts and/or solvates, a method for preparing these compounds and use as therapeutic compounds.

### BACKGROUND OF INVENTION

At least five physiological mechanisms have been proposed to explain referred pain: (1) pain by excess of nociception, (2) neuropathic pain, (3) activity in sympathetic nerves, (4) idiopathic pain, and (5) psychogenic pain.

Pain by excess of nociception is the most frequent mechanism and results from tissue damage that causes an excess of painful impulses transmitted by an intact nervous system. It results in localized pain of non-neurological topography, with an inflammatory or mechanical schedule.

It may also be of visceral origin. It then appears less well localized, deep, sometimes accompanied by nausea and vomiting.

With a less known mechanism, visceral pain represents a major clinical problem and a common cause for seeking medical attention for both men and women. Visceral pain is experienced by 40% of the population, and 28% of cancer patients suffer from pain arising from intra-abdominal metastasis or from treatment. The recent growth in interest by researchers and clinicians in pain originating from internal organs reflects an important paradigm shift in the awareness of the magnitude and impact of visceral pain disorders.

It is well recognized that visceral hypersensitivity can occur due to (1) sensitization of primary sensory afferents innervating the viscera, (2) hyperexcitability of spinal ascending neurons (central sensitization) receiving synaptic input from the viscera, and (3) dysregulation of descending pathways that modulate spinal nociceptive transmission.

Although with the advances of medical science, the acute pain associated with infection and disease can be correctly diagnosed and treated, many chronic pain syndromes still remain a challenge for clinicians. Such chronic pain in the viscera is observed in functional bowel disorders (e.g., noncardiac chest pain, chronic idiopathic dyspepsia, functional abdominal pain, irritable bowel syndrome; IBS, Crohn's disease) and chronic pelvic pain (e.g., chronic interstitial cystitis, painful bladder syndrome or endometriosis) that are multifaceted problems and still poorly understood.

Functional bowel disorders and chronic pelvic pain represent unexplained symptoms that have no readily identifiable infectious, anatomical, or metabolic basis, it is however known that visceral pain may be induced by drugs causing inflammation in viscera.

For example, Cyclophosphamide, an alkylating agent, widely used in malignant and a variety of inflammatory diseases, is known to produce toxic effects on the bladder wall through its main toxic metabolite acrolein, leading to acute haemorrhagic cystitis (HC). Bladder complications besides acute haemorrhagic cystitis include bladder fibrosis and carcinoma.

Several channels are important to visceral pain: transient receptor potential vanilloid-1 (TRPV-1), ASIC3 channels and sodium channels (NaV) particularly those that are tetrodotoxin resistant (NAV 1.7, 1.8 and 1.9), and calcium channels. Certain receptors downmodulate pain: the gamma aminobutyric acid-B (GABA-B) channels, kappa and mu opioid receptors, and somatostatin receptors. These channels and receptors are potential targets for novel analgesics to treat visceral pain.

Visceral pain is included with somatic and nociceptive pain in most clinical trials; as a result, it is difficult to determine the appropriate drug choices for visceral pain as a phenotype. The fact that there are some differences between somatic and visceral pain neurotransmission, neurotransmitters, channels, and receptors suggests that there may in fact be real differences in responses to analgesics. For example, the use of potent opioids for inflammatory bowel disease has been associated with higher morbidity and mortality which is not reported for various types of somatic pain. In a similar fashion, certain NSAIDs have been associated with poorer outcomes in inflammatory bowel disease but not for somatic pain. On the other hand, octreotide improves colic and symptoms from bowel obstruction better than anticholinergics; potent opioids actually may worsen colic.

Thus, there is still a need for an effective approach for the management of visceral pain including both somatic and nociceptive pain.

The goal of this invention is to provide new nicotinamide di-nucleotide derivatives for use in the management of visceral pain. Surprisingly, the applicant found that nicotinamide di-nucleotide derivatives of the invention are well tolerated and displayed significant anti-nociceptive activity in CYP-induced cystitis model.

### SUMMARY

This invention thus relates to a compound of formula I

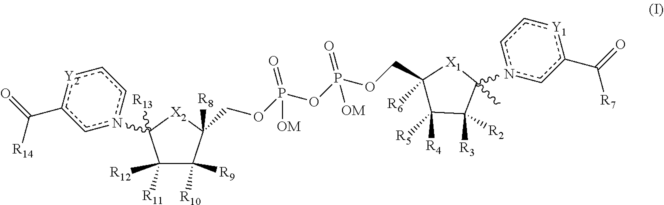

or pharmaceutically acceptable salts and/or solvates thereof, wherein:
X₁ and X₂ are independently selected from O, CH₂, S, Se, CHF, CF₂ and C═CH₂;
R₁ and R₁₃ are independently selected from H, azido, cyano, C1-C8 alkyl, C1-C8 thio-alkyl, C1-C8 heteroalkyl and OR, wherein R is selected from H and C1-C8 alkyl;
R₂, R₃, R₄, R₅, R₉, R₁₀, R₁₁, R₁₂ are independently selected from H, halogen, azido, cyano, hydroxyl, C1-C12 alkyl, C1-C12 thio-alkyl, C1-C12 heteroalkyl, C1-C12 haloalkyl and OR, wherein R is selected from H, C1-C12 alkyl, C(O)(C1-C12)alkyl, C(O)NH(C1-C12)alkyl, C(O)O(C1-C12)alkyl, C(O)aryl, C(O)(C1-C12)alkyl aryl, C(O)NH(C1-C12)alkyl aryl, C(O)O(C1-C12)alkyl aryl or C(O)CHR_{AA}NH₂, wherein R_{AA} is a side chain selected from a proteinogenic amino acid;
R₆ and R₈ are independently selected from H, azido, cyano, C1-C8 alkyl and OR; wherein R is selected from H and C1-C8 alkyl;
R₇ and R₁₄ are independently selected from H, OR, NHR, NRR′, NH—NHR, SH, CN, N₃ and halogen, wherein R and R′ are each independently selected from H, C1-C8 alkyl, (C1-C8)alkyl aryl;
Y₁ and Y₂ are independently selected from CH, CH₂, C(CH₃)₂ or CCH₃;
M is selected from H or a suitable counterion;
[symbol] represents a single or a double bound depending on Y₁ and Y₂; and
[symbol] represents the alpha or beta anomer depending on the position of R₁ and R₁₃.

The present invention further relates to the compound of formula I as described herein above for use as a medicament.

According to one embodiment, M is an internal or external counterion.

According to one embodiment, X₁ and X₂ each independently represents an oxygen.

According to one embodiment, R₇ and R₁₄ each independently represents a NH₂.

According to one embodiment, R₁ and/or R₁₃ each independently represents a hydrogen.

According to one embodiment, R₆ and/or R₈ each independently represents a hydrogen.

According to one embodiment, R₃, R₄, R₁₀, R₁₁ are identical and represent each a hydrogen.

According to one embodiment, R₂, R₅, R₉ and R₁₂ are identical and represent each a hydroxyl.

According to one embodiment, Y₁ and Y₂ each independently represents a CH.

According to one embodiment, Y₁ and Y₂ each independently represents a CH₂. According to one embodiment, the compound according to the invention is selected from compounds of formula I-A to I-F:

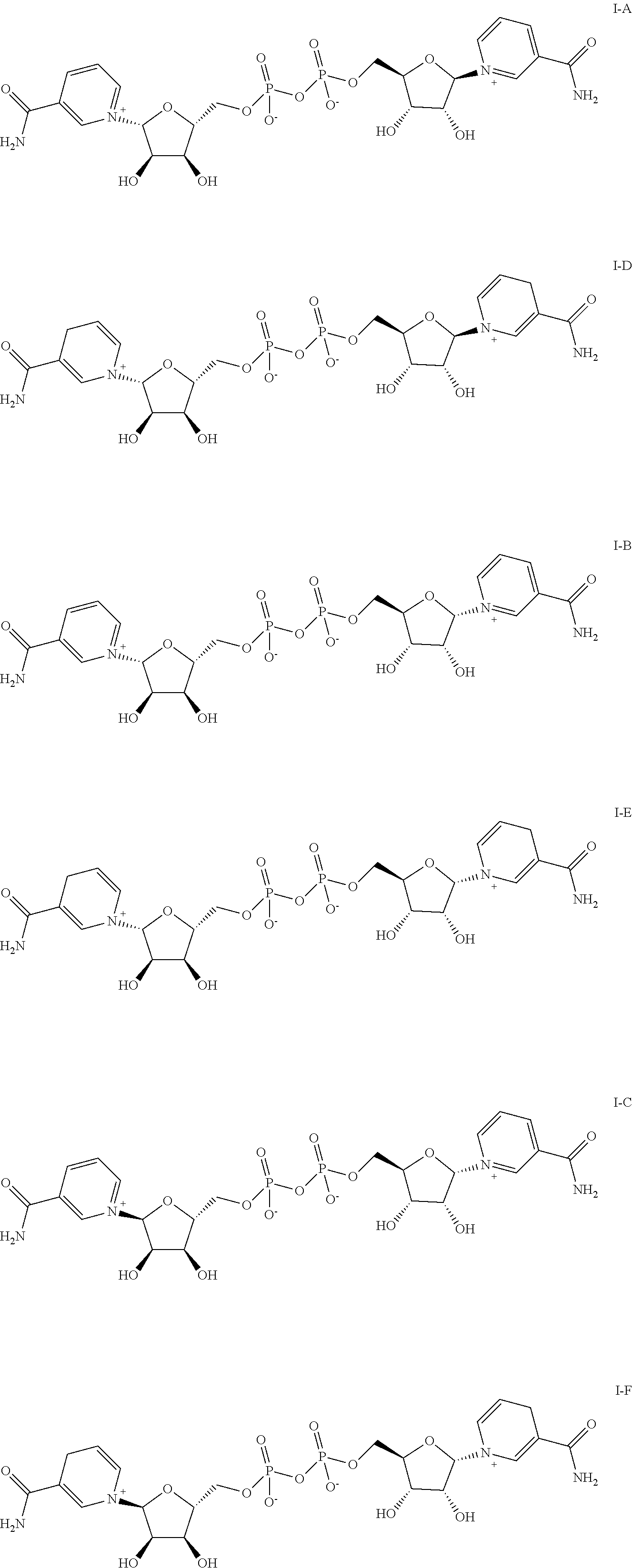

According to one embodiment, the compound is of formula I-A, I-B or I-C.

The present invention further relates to a compound of formula I′

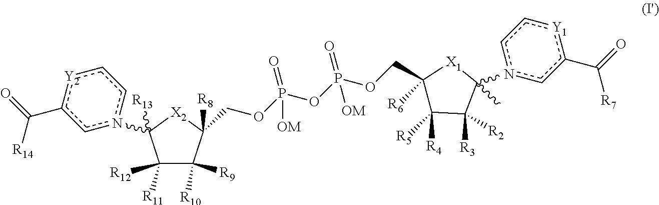

or pharmaceutically acceptable salts and/or solvates thereof, wherein:
   X₁ and X₂ are independently selected from O, CH₂, S, Se, CHF, CF₂ and C═CH₂;
   R₁ and R₁₃ are independently selected from H, azido, cyano, C1-C8 alkyl, C1-C8 thio-alkyl, C1-C8 heteroalkyl and OR; wherein R is selected from H and C1-C8 alkyl;
   R₂, R₃, R₄, R₅, R₉, R₁₀, R₁₁, R₁₂ are independently selected from H, halogen, azido, cyano, hydroxyl, C1-C12 alkyl, C1-C12 thio-alkyl, C1-C12 heteroalkyl, C1-C12 haloalkyl and OR; wherein R is selected from H, C1-C12 alkyl, C(O)(C1-C12)alkyl, C(O)NH(C1-C12)alkyl, C(O)O(C1-C12)alkyl, C(O)aryl, C(O)(C1-C12)alkyl aryl, C(O)NH(C1-C12)alkyl aryl, C(O)O(C1-C12)alkyl aryl or C(O)CHR_{AA}NH₂, wherein R_{AA} is a side chain selected from a proteinogenic amino acid;
   R₆ and R₈ are independently selected from H, azido, cyano, C1-C8 alkyl and OR; wherein R is selected from H and C1-C8 alkyl;
   R₇ and R₁₄ are independently selected from H, OR, NHR, NRR′, NH—NHR, SH, CN, N₃ and halogen; wherein R and R′ are each independently selected from H, C1-C8 alkyl, (C1-C8)alkyl aryl;
   Y₁ and Y₂ are independently selected from CH, CH₂, C(CH₃)₂ or CCH₃;
   M is selected from H or a suitable counterion;
   [symbol] represents a single or a double bound depending on Y₁ and Y₂; and
   [symbol] represents the alpha or beta anomer depending on the position of R₁ and R₁₃,
with the proviso that when: X₁ and X₂ are oxygen; R₁, R₃, R₄, R₆, R₅, R₁₀, Ru, and R₁₃ are hydrogen; R₂, R₅, R₉ and R₁₂ are hydroxyl; R₇ and R₁₄ are NH₂; and Y₁ and Y₂ are independently selected from CH or CH₂,
then at least one of [symbol] represent the alpha anomer.

The present invention further relates to a pharmaceutical composition comprising at least one compound for use according to the invention or at least one compound according to the invention, and at least one pharmaceutically acceptable carrier.

The present invention further relates to a food composition comprising at least one compound for use according to the invention or at least one compound according to the invention, and at least one acceptable carrier and/or diluent.

The present invention further relates to a cosmetic composition comprising at least one compound for use according to any one compound for use according to the invention or at least one compound according to the invention, and at least one acceptable carrier and/or diluent.

According to one embodiment, the compound for use according to the invention or the compound according to the invention, are for use in the treatment of pain.

According to one embodiment, the compound for use according to the invention or the compound according to the invention, are for use the treatment of an antineoplastic-induced cardiotoxicity or sickle cell disease.

The present invention further relates to a method for preparing compounds of formula I or of formula I′, comprising the following steps:
1) mono-phosphorylation of a compound of formula X,

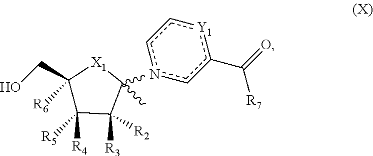

wherein:
X₁, R₁, R₂, R₃, R₄, R₅, R₆, R₇, Y₁, [symbol] and [symbol] are as defined for formula I or formula I′,
to give compound of formula XI,

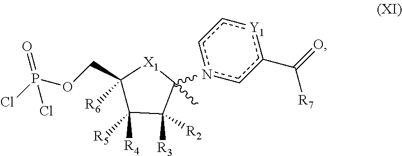

wherein:
X₁, R₁, R₂, R₃, R₄, R₅, R₆, R₇, Y₁, [symbol] and [symbol] are as defined for formula I or formula I′;
2) hydrolysis of compound of formula XI obtained in step 1), to give compound of formula XII

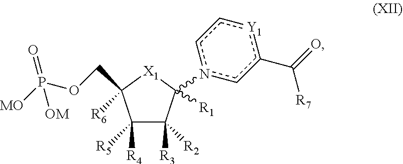

wherein:
X₁, R₁, R₂, R₃, R₄, R₅, R₆, R₇, Y₁, [symbol] and [symbol] are as defined for formula I or formula I′;
3) reacting compound of formula XII obtained in step 2) with compound of formula XIII,

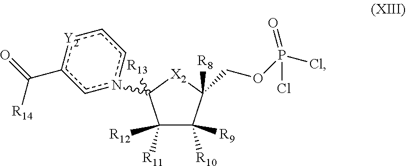

obtained as described in step 1) and wherein:
X₂, R₈, R₉, R₁₀, R₁₁, R₁₂, R₁₃, R₁₄, Y₂, [symbol] and [symbol] are as defined for formula I or formula I′,
to give compound of formula I or formula I′.

According to one embodiment, the method further comprises a step of reducing the compound of formula I or formula I′ obtained in step 3), to give the compound of formula I or formula I′, wherein Y₁ and Y₂ each independently represents a CH₂.

### Definitions

In the present invention, the following terms have the following meanings.

Unless indicated otherwise, the nomenclature of substituents that are not explicitly defined herein are arrived at by naming the terminal portion of the functionality followed by the adjacent functionality toward the point of attachment.
“alkyl” by itself or as part of another substituent refers to a hydrocarbyl radical of Formula CnH2n+1 wherein n is a number greater than or equal to 1. Generally, alkyl groups of this invention comprise from 1 to 12 carbon atoms, preferably from 1 to 8 carbon atoms, more preferably from 1 to 6 carbon atoms, still more preferably 1 to 2 carbon atoms. Alkyl groups may be linear or branched and may be substituted as indicated herein.
Suitable alkyl groups include methyl, ethyl, n-propyl, i-propyl, n-butyl, i-butyl, s-butyl and t-butyl, pentyl and its isomers (e.g. n-pentyl, iso-pentyl), hexyl and its isomers (e.g. n-hexyl, iso-hexyl), heptyl and its isomers (e.g. n-heptyl, iso-heptyl), octyl and its isomers (e.g. n-octyl, iso-octyl), nonyl and its isomers (e.g. n-nonyl, iso-nonyl), decyl and its isomers (e.g. n-decyl, iso-decyl), undecyl and its isomers, dodecyl and its isomers. Preferred alkyl groups include methyl, ethyl, n-propyl, i-propyl, n-butyl, i-butyl, s-butyl and t-butyl. Cx-Cy-alkyl refer to alkyl groups which comprise from x to y carbon atoms.
“alkoxy” refers to an alkyl group as defined above which is attached to another moiety by an oxygen atom. Examples of alkoxy groups include methoxy, isopropoxy, ethoxy, tert-butoxy, and the like. Alkoxy groups may be optionally substituted with one or more substituents. Alkoxy groups included in compounds of this invention may be optionally substituted with a solubilizing group.
“aryl” as used herein refers to a polyunsaturated, aromatic hydrocarbyl group having a single ring (i.e. phenyl) or multiple aromatic rings fused together (e.g. naphtyl) or linked covalently, typically containing 5 to 12 atoms; preferably 6 to 10, wherein at least one ring is aromatic. The aromatic ring may optionally include one to two additional rings (either cycloalkyl, heterocyclyl or heteroaryl) fused thereto. Aryl is also intended to include the partially hydrogenated derivatives of the carbocyclic systems enumerated herein. Examples of aryl comprise phenyl, biphenylyl, biphenylenyl, 5- or 6-tetralinyl, naphthalen-1- or -2-yl, 4-, 5-, 6 or 7-indenyl, 1-2-, 3-, 4- or 5-acenaphtylenyl, 3-, 4- or 5-acenaphtenyl, 1- or 2-pentalenyl, 4- or 5-indanyl, 5-, 6-, 7- or 8-tetrahydronaphthyl, 1,2,3,4-tetrahydronaphthyl, 1,4-dihydronaphthyl, 1-, 2-, 3-, 4- or 5-pyrenyl.
“Alkylaryl” refers to an aryl group substituted by an alkyl group: alkyl-aryl-.
“Amino acid” refers to an alpha-amino carboxylic acid, i. e. a molecule comprising a carboxylic acid functional group and an amine functional group in the alpha position of the carboxylic acid group, for example a proteinogenic amino acid or a non-proteinogenic amino acid such as 2 aminoisobutyric acid.
“Proteinogenic amino acid” refers to an amino acid that is incorporated into proteins during the translation of messenger RNA by ribosomes in living organisms, i.e. Alanine (ALA), Arginine (ARG), Asparagine (ASN), Aspartate (ASP), Cysteine (CYS), Glutamate (Glutamic acid) (GLU), Glutamine (GLN), Glycine (GLY), Histidine (HIS), Isoleucine (ILE), Leucine (LEU), Lysine (LYS), Methionine (MET), Phenylalanine (PHE), Proline (PRO), Pyrrolysine (PYL), Selenocysteine (SEL), Serine (SER), Threonine (THR), Tryptophan (TRP), Tyrosine (TYR) or Valine (VAL).
“halogen” “halo” or “halogen” means fluoro, chloro, bromo, or iodo. Preferred halo groups are fluoro and chloro.
“haloalkyl” alone or in combination, refers to an alkyl radical having the meaning as defined above wherein one or more hydrogens are replaced with a halogen as defined above. Examples of such haloalkyl radicals include chloromethyl, 1-bromoethyl, fluoromethyl, difluoromethyl, trifluoromethyl, 1,1,1-trifluoroethyl and the like. C_{x-y}-haloalkyl and Cx-Cy-alkyl refer to alkyl groups which comprise from x to y carbon atoms. Preferred haloalkyl groups are difluoromethyl, trifluoromethyl.
“Heteroalkyl” refers to an alkyl group as defined hereinabove wherein one or more carbon atoms are replaced by a heteroatom selected from oxygen, nitrogen and sulphur atoms. In heteroalkyl groups, the heteroatoms are bond along the alkyl chain only to carbon atoms, i.e., each heteroatom is separated from any other heteroatom by at least one carbon atom. However, the nitrogen and sulfur heteroatoms may optionally be oxidized and the nitrogen heteroatoms may optionally be quaternized. An heteroalkyl is bond to another group or molecule only through a carbon atom, i.e., the bounding atom is not selected among the heteroatoms included in the heteroalkyl group.
“pharmaceutically acceptable salts” include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine, 2-(diethylamino)ethanol, ethanolamine, morpholine, 4-(2-hydroxyethyl)morpholine and zinc salts. Hemisalts of acids and bases may also be formed, for example, hemisulphate and hemicalcium salts. Preferred, pharmaceutically acceptable salts include hydrochloride/chloride, hydrobromide/bromide, bisulphate/sulphate, nitrate, citrate, and acetate.
Pharmaceutically acceptable salts may be prepared by one or more of these methods:
(i) by reacting the compound with the desired acid;
(ii) by reacting the compound with the desired base;
(iii) by removing an acid- or base-labile protecting group from a suitable precursor of the compound or by ring-opening a suitable cyclic precursor, for example, a lactone or lactam, using the desired acid; or
(iv) by converting one salt of the compound to another by reaction with an appropriate acid or by means of a suitable ion exchange column.
All these reactions are typically carried out in solution. The salt, may precipitate from solution and be collected by filtration or may be recovered by evaporation of the solvent. The degree of ionization in the salt may vary from completely ionized to almost non-ionized.
“pharmaceutically acceptable” means approved or approvable by a regulatory agency or listed in recognized pharmacopeia for use in animals, and more preferably in humans. It can be material which is not biologically or otherwise undesirable, i.e. the material can be administered to an individual without causing any undesirable biological effects or interacting in a deleterious manner with any of the components of the composition in which it is contained.
“solvate” is used herein to describe a molecular complex comprising the compound of the invention and one or more pharmaceutically acceptable solvent molecules, for example, ethanol.
term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen, alkyl or aryl groups as defined above, hydroxyl, alkoxy group as defined above, nitro, thiol, heterocycloalkyl groups, heteroaryl groups, cyano, cycloalkyl groups as defined above, as well as a solubilizing group, —NRR′, —NR—CO—R′, —CONRR′, —SO₂NRR′ group wherein R and R′ are each independently selected from hydrogen, alkyl, cycloalkyl, aryl, heterocycloalkyl or heteroaryl groups as defined above.
The bonds from an asymmetric carbon may be depicted herein using a solid wedge ( [symbol] ), or a dotted wedge ( [symbol] ) or a zigzag line ( [symbol] ).

### DETAILED DESCRIPTION

### Compounds

This invention relates to a compound of formula I or pharmaceutically acceptable salts and/or solvates thereof:

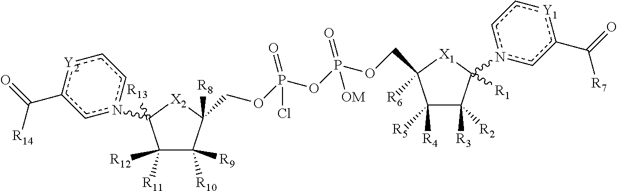

wherein:
X₁ and X₂ are independently selected from 0, CH₂, S, Se, CHF, CF₂ and C═CH₂; R₁ and R₁₃ are independently selected from H, azido, cyano, C1-C8 alkyl, C1-C8 thio-alkyl, C1-C8 heteroalkyl and OR; wherein R is selected from H and C1-C8 alkyl;
R₂, R₃, R₄, R₅, R₉, R₁₀, R₁₁, R₁₂ are independently selected from H, halogen, azido, cyano, hydroxyl, C1-C12 alkyl, C1-C12 thio-alkyl, C1-C12 heteroalkyl, C1-C12 haloalkyl and OR, wherein R is selected from H, C1-C12 alkyl, C(O)(C1-C12)alkyl, C(O)NH(C1-C12)alkyl, C(O)O(C1-C12)alkyl, C(O)aryl, C(O)(C1-C12)alkyl aryl, C(O)NH(C1-C12)alkyl aryl, C(O)O(C1-C12)alkyl aryl or C(O)CHR_{AA}NH₂, wherein R_{AA} is a side chain selected from a proteinogenic amino acid;
R₆ and R₈ are independently selected from H, azido, cyano, C1-C8 alkyl and OR; wherein R is selected from H and C1-C8 alkyl;
R₇ and R₁₄ are independently selected from H, OR, NHR, NRR′, NH—NHR, SH, CN, N₃ and halogen, wherein R and R′ are each independently selected from H, C1-C8 alkyl, (C1-C8)alkyl aryl;
Y₁ and Y₂ are independently selected from CH, CH₂, C(CH₃)₂ or CCH₃;
M is selected from H or a suitable counterion;
[symbol] represents a single or a double bound depending on Y₁ and Y₂; and
[symbol] represents the alpha or beta anomer depending on the position of R₁ and R₁₃.

According to one embodiment, M is an internal or external counterion.

According to an embodiment, preferred compounds of general Formula I are those wherein X₁ and X₂ are independently selected from O, CH₂, S.

According to one embodiment, R₇ and R₁₄ are independently selected from H, OR, NHR and NRR′ wherein R and R′ are independently selected from H, C1-C8 alkyl, (C1-C8)alkyl aryl. According to one embodiment, R₇ and R₁₄ are NHR wherein R is selected from H, C1-C8 alkyl, (C1-C8)alkyl aryl.

According to one embodiment, R₂, R₃, R₄, R₅, R₉, R₁₀, R₁₁, R₁₂ are independently selected from H, halogen, hydroxyl, C1-C12 alkyl and OR, wherein R is as described herein above. According to a preferred embodiment, R₂, R₃, R₄, R₅, R₉, R₁₀, R₁₁, R₁₂ are independently selected from H, hydroxyl and OR, wherein R is as described herein above.

According to an embodiment, preferred compounds of general Formula I are those wherein, R₂, R₃, R₄, R₅, R₉, R₁₀, R₁₁, R₁₂ are independently selected from H and OH.

According to one embodiment, R₂ and R₃ are identical. According to one embodiment, R₂ and R₃ are identical and represent each a OH. According to one embodiment, R₂ and R₃ are identical and represent each hydrogen.

According to a preferred embodiment, R₂ and R₃ are different. According to a preferred embodiment, R₂ is hydrogen and R₃ is a OH. According to a more preferred embodiment, R₂ is a OH and R₃ is hydrogen.

According to one embodiment, R₄ and R₅ are identical. According to one embodiment, R₄ and R₅ are identical and represent each a OH. According to one embodiment, R₄ and R₅ are identical and represent each hydrogen.

According to a preferred embodiment, R₄ and R₅ are different. According to a preferred embodiment, R₄ is a OH and R₅ is hydrogen. According to a more preferred embodiment, R₄ is hydrogen and R₅ is a OH.

According to one embodiment, R₃ and R₄ are identical. According to one embodiment, R₃ and R₄ are identical and represent each a OH. According to one embodiment, R₃ and R₄ are identical and represent each hydrogen.

According to a preferred embodiment, R₃ and R₄ are different. According to a preferred embodiment, R₃ is a OH and R₄ is hydrogen. According to a more preferred embodiment, R₃ is hydrogen and R₄ is a OH

According to one embodiment, R₂ and R₅ are different. According to one embodiment, R₂ is hydrogen and R₅ is a OH. According to one embodiment, R₂ is a OH and R₅ is hydrogen.

According to a preferred embodiment, R₂ and R₅ are identical. According to a preferred embodiment, R₂ and R₅ are identical and represent each hydrogen. According to a more preferred embodiment, R₂ and R₅ are identical and represent each a OH.

According to one embodiment, R₉ and R₁₀ are identical. According to one embodiment, R₉ and R₁₀ are identical and represent each a OH. According to one embodiment, R₉ and R₁₀ are identical and represent each hydrogen.

According to a preferred embodiment, R₉ and R₁₀ are different. According to a preferred embodiment, R₉ is hydrogen and R₁₀ is a OH. According to a more preferred embodiment, R₉ is a OH and R₁₀ is hydrogen.

According to one embodiment, R₁₁ and R₁₂ are identical. According to one embodiment, R₁₁ and R₁₂ are identical and represent each a OH. According to one embodiment, R₁₁ and R₁₂ are identical and represent each hydrogen.

According to a preferred embodiment, R₁ and R₁₂ are different. According to a preferred embodiment, R₁₁ is a OH and R₁₂ is hydrogen. According to a more preferred embodiment, R₁₁ is hydrogen and R₁₂ is a OH.

According to one embodiment, R₁₀ and R₁₁ are different. According to one embodiment, R₁₀ is hydrogen and R₁₁ is a OH. According to one embodiment, R₁₀ is a OH and R₁₁ is hydrogen.

According to a preferred embodiment, R₁₀ and R₁₁ are identical. According to a preferred embodiment, R₁₀ and R₁₁ are identical and represent each a OH. According to a more preferred embodiment, R₁₀ and R₁₁ are identical and represent each hydrogen.

According to one embodiment, R₉ and R₁₂ are different. According to one embodiment, R₉ is hydrogen and R₁₂ is a OH. According to one embodiment, R₉ is a OH and R₁₂ is hydrogen.

According to a preferred embodiment, R₉ and R₁₂ are identical. According to a preferred embodiment, R₉ and R₁₂ are identical and represent each hydrogen. According to a more preferred embodiment, R₉ and R₁₂ are identical and represent each a OH.

According to one embodiment, Y₁ is CH. According to one embodiment, Y₁ is CH₂.

According to one embodiment, Y₂ is CH. According to one embodiment, Y₂ is CH₂.

According to one embodiment, X₁ and X₂ are different and are selected from the group as described above. According to one embodiment, X₁ and X₂ are identical and are selected from the group as described above.

According to an embodiment, preferred compounds of general Formula I are those wherein X₁ and X₂ each independently represents an Oxygen.

According to an embodiment, preferred compounds of general Formula I are those wherein X₁ and X₂ are identical and represent each an Oxygen.

According to a preferred embodiment, among the compounds of formula I, the present invention is directed to compounds having the following formula II:

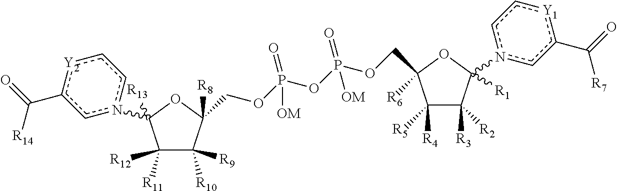

or pharmaceutically acceptable salts and/or solvates thereof, wherein R₁, R₂, R₃, R₄, R₅, R₆, R₇, R₉, R₈, R₉, R₁₀, R₁₁, R₁₂, R₁₃, R₁₄, Y₁, Y₂, M, [symbol] and [symbol] are as described above.

[symbol]

According to one embodiment, R₇ and R₁₄ are different and are selected from the group as described above. According to one embodiment, R₇ and R₁₄ are identical and are selected from the group as described above.

According to an embodiment, preferred compounds of general Formula I are those wherein R₇ and R₁₄ each independently represents a NH₂.

According to an embodiment, preferred compounds of general Formula I are those wherein R₇ and R₁₄ are identical and represent each a NH₂.

According to a preferred embodiment, among the compounds of formula I, the present invention is directed to compounds having the following formula III:

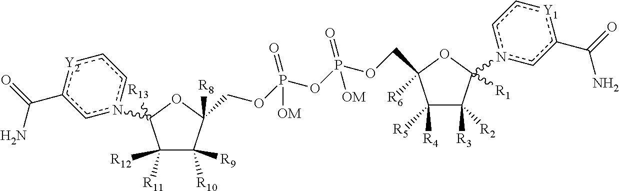

or pharmaceutically acceptable salt and/or solvates thereof, wherein R₁, R₂, R₃, R₄, R₅, R₆, R₈, R₉, R₁₀, R₁₁, R₁₂, R₁₃, Y₁, Y₂, M, [symbol] and [symbol] are as described above.

According to one embodiment, R₁ and R₁₃ are different and are selected from the group as described above. According to one embodiment, R₁ and R₁₃ are identical and are selected from the group as described above.

According to an embodiment, preferred compounds of general Formula I are those wherein R₁ and R₁₃ each independently represents a hydrogen.

According to an embodiment, preferred compounds of general Formula I are those wherein R₁ and R₁₃ are identical and represent each a hydrogen.

According to a preferred embodiment, among the compounds of formula I, the present invention is directed to compounds having the following formula IV:

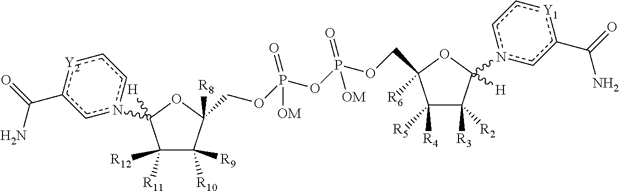

or pharmaceutically acceptable salt and/or solvates thereof, wherein R₂, R₃, R₄, R₅, R₆, R₇, R₈, R₉, R₁₀, R₁₁, R₁2, Y₁, Y2, M,

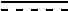

and

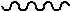

are as described above.

According to one embodiment, R₆ and R₈ are different and are selected from the group as described above. According to one embodiment, R₆ and R₈ are identical and are selected from the group as described above.

According to an embodiment, preferred compounds of general Formula I are those wherein R₆ and R₈ each independently represents a hydrogen.

According to an embodiment, preferred compounds of general Formula I are those wherein R₆ and R₈ are identical and represent each a hydrogen.

According to a preferred embodiment, among the compounds of formula I, the present invention is directed to compounds having the following formula V:

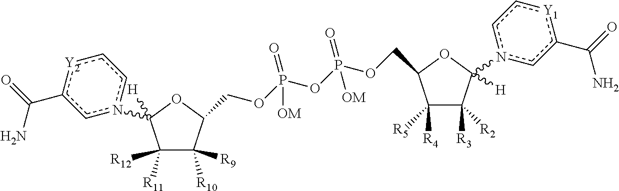

or pharmaceutically acceptable salt and/or solvates thereof, wherein R₂, R₃, R₄, R₅, R₉, R₁₀, R₁₁, R₁₂, Y₁, Y₂, M, [symbol] and [symbol] are as described above.

According to one embodiment, R₃, R₄, R₁₀ and R₁₁ are different and are selected from the group as described above. According one embodiment, R₃, R₄, R₁₀ and R₁₁ are identical and are selected from the group as described above.

According to an embodiment, preferred compounds of general Formula I are those wherein R₃, R₄, R₁₀ and R₁₁ each independently represents a hydrogen.

According to an embodiment, preferred compounds of general Formula I are those wherein R₃, R₄, R₁₀, R₁₁ are identical and represent each a hydrogen.

According to a preferred embodiment, among the compounds of formula I, the present invention is directed to compounds having the following formula VI:

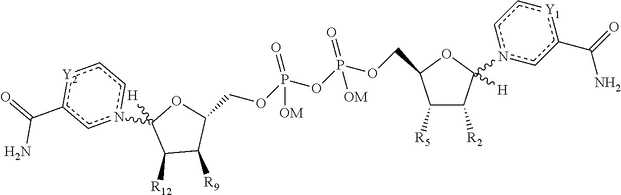

or pharmaceutically acceptable salt and/or solvates thereof, wherein R₂, R₅, R₉, R₁₂, Y₁, Y₂, M, [symbol] and [symbol] are as described above.

According to one embodiment, R₂, R₅, R₉ and R₁₂ are different and are selected from the group as described above. According one embodiment, R₂, R₅, R₉ and R₁₂ are identical and are selected from the group as described above.

According to an embodiment, preferred compounds of general Formula I are those wherein R₂, R₅, R₉ and R₁₂ each independently represents a OH.

According to an embodiment, preferred compounds of general Formula I are those wherein R₂, R₅, R₉, R₁₂ are identical and represent each a OH.

According to a preferred embodiment, among the compounds of formula I, the present invention is directed to compounds having the following formula VII:

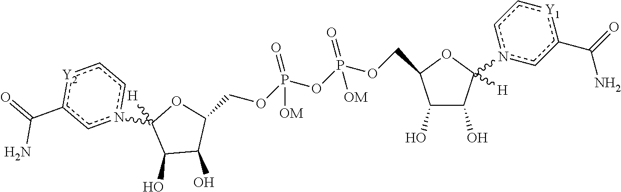

or pharmaceutically acceptable salt and/or solvates thereof, wherein Y₁, Y₂, M, [symbol] and [symbol] are as described above.

According to one embodiment, Y₁ and Y₂ are different. According to a preferred embodiment, Y₁ and Y₂ are identical.

According to an embodiment, preferred compounds of general Formula I are those wherein Y₁ and Y₂ each independently represents a CH.

According to an embodiment, preferred compounds of general Formula I are those wherein Y₁ and Y₂ are identical and represent each a CH.

According to a preferred embodiment, among the compounds of formula I, the present invention is directed to compounds having the following formula VIII:

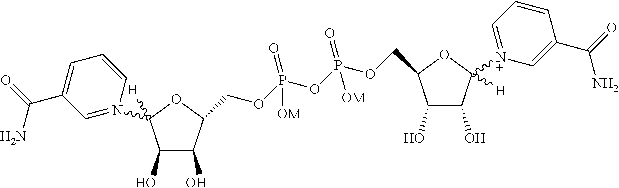

or pharmaceutically acceptable salt and/or solvates thereof, wherein M and [symbol] are as described above.

[symbol]

According to an embodiment, preferred compounds of general Formula I are those wherein Y₁ and Y₂ each independently represents a CH₂.

According to an embodiment, preferred compounds of general Formula I are those wherein Y_{C} and Y₂ are identical and represent each a CH₂.

According to a preferred embodiment, among the compounds of formula I, the present invention is directed to compounds having the following formula IX:

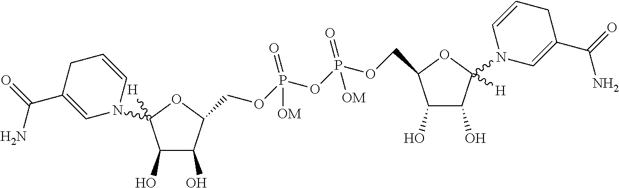

or pharmaceutically acceptable salt and/or solvates thereof, wherein M and [symbol] are as described above. According to one embodiment, preferred compounds of the invention are compounds I-A to I-F, listed in table 1:

TABLE 1

| Cpd n° | |
|---|---|
| (anomers) | Structure |
| I-A (beta, beta) | 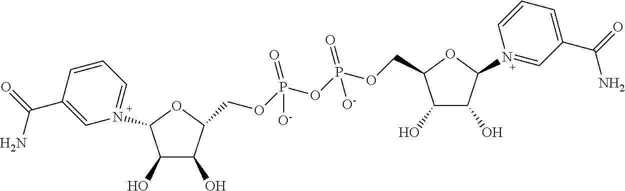 |
| I-B (beta, alpha) | 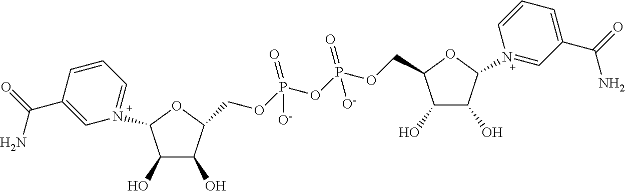 |
| I-C (alpha, alpha) | 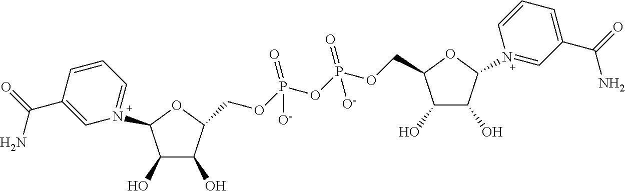 |
| I-D (beta, beta) | 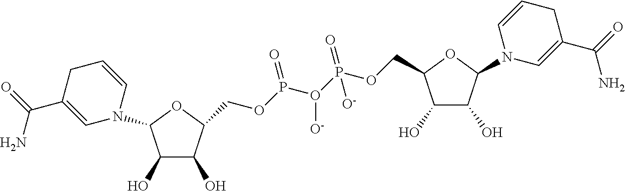 |
| I-E (beta, alpha) | 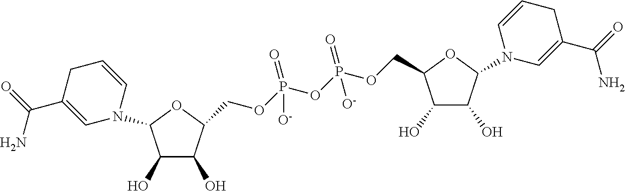 [SMILES: NC(=O)C1=CN([C@@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |
| I-F (alpha, alpha) | 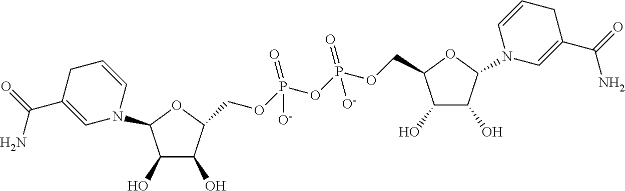 [SMILES: NC(=O)C1=CN([C@H]2O[C@H](COP(=O)([O-])OP(=O)([O-])OC[C@H]3O[C@H](N4C=CCC(C(N)=O)=C4)[C@H](O)[C@@H]3O)[C@@H](O)[C@H]2O)C=CC1] |

According to one embodiment, preferred compound of the invention are compounds of formula I-A-I-C.

According to one embodiment, preferred compound of the invention is compound of formula I-A.

According to one embodiment, preferred compound of the invention is compound of formula I-B.

According to one embodiment, preferred compound of the invention is compound of formula I-C.

According to another embodiment, preferred compound of the invention is compound of formula I-D.

According to another embodiment, preferred compound of the invention is compound of formula I-E.

According to another embodiment, preferred compound of the invention is compound of formula I-F.

The invention also relates to a compound of formula I′ or pharmaceutically acceptable salts and/or solvates thereof:

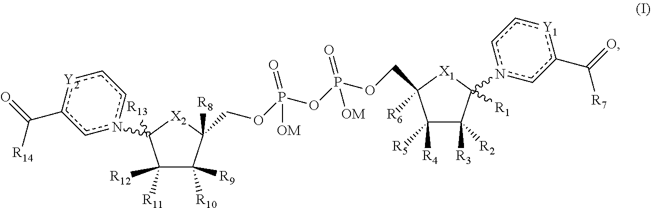

wherein X₁, X₂, R₁, R₂, R₃, R₄, R₅, R₆, R₇, R₈, R₉, R₁₀, R₁₁, R₁₂, R₁₃, R₁₄, Y₁, Y₂, M, [symbol] and [symbol] are as described herein above for the compound of formula I,

provided that when: X₁ and X₂ are oxygen; R₁, R₃, R₄, R₆, R₈, R₁₀, Ru, and R₁₃ are hydrogen; R₂, R₅, R₉ and R₁₂ are hydroxyl; R₇ and R₁₄ are NH₂; and Y₁ and Y₂ are independently selected from CH or CH₂,

then at least one of [symbol] represent the alpha anomer.

According to the invention, M can be an internal or external counterion.

In this particular embodiment, following compounds are excluded from the formula I′:

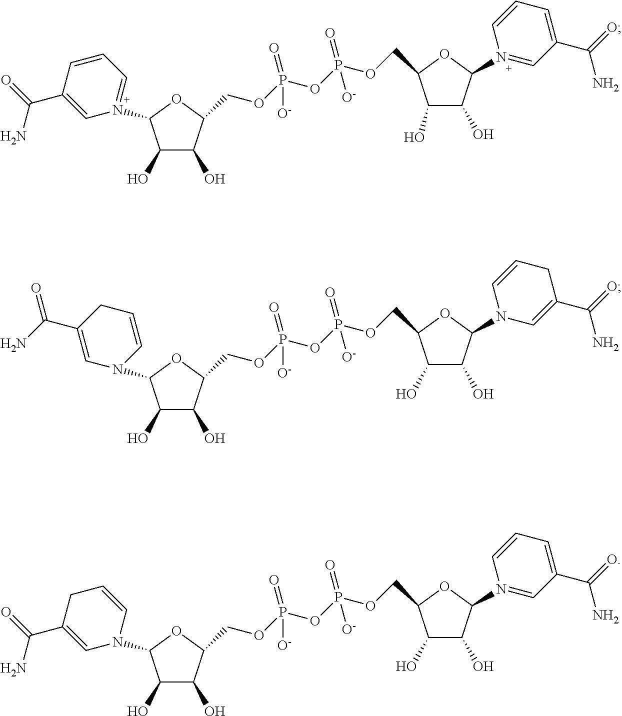

### Process

In another aspect, the invention relates to a method for preparing compounds of formula I or formula I′ as described above.

In particular, compounds of formula I or formula I′ disclosed herein can be prepared as described below from substrates X-XIII. It will be understood by one ordinary skilled in the art that these schemes are in no way limiting and that variations of detail can be made without departing from the spirit and scope of the present invention.

According to one embodiment, the invention relates to a method for preparing the compound of formula I or formula I′ as described herein above.

The method first involves the mono-phosphorylation of a compound of formula X, in the presence of phosphoryl chloride in a trialkyl phosphate, to give the phophorodichloridate compound XI,

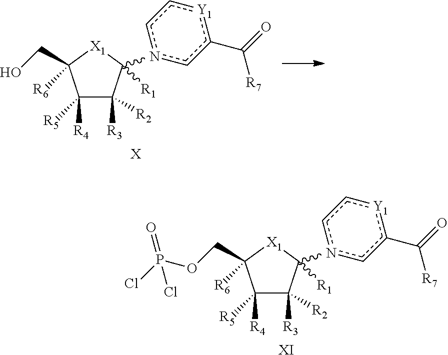

wherein X₁, R₁, R₂, R₃, R₄, R₅, R₆, R₇, Y₁, [symbol] and [symbol] are as described herein for formula I or formula I′.

In a second step the hydrolysis of the phophorodichloridate XI obtained in the first step give the phosphate compound of formula XII,

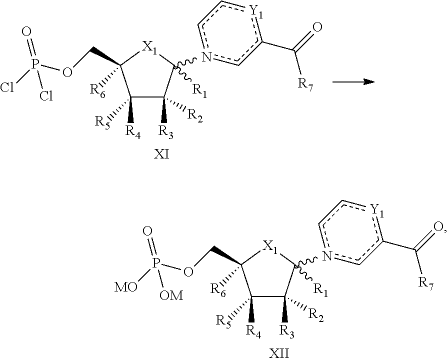

wherein X₁, R₁, R₂, R₃, R₄, R₅, R₆, R₇, Y₁, M, [symbol] and [symbol] are as described herein for formula I or formula I′.

The phosphate compound of formula XII obtained in the second step is then reacted, with a phophorodichloridate compound of formula XIII obtained as described in the first step,

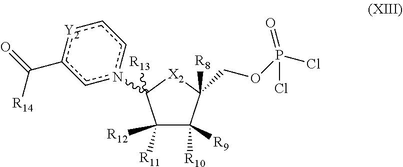

wherein X₂, R₈, R₉, R₁₀, R₁₁, R₁₂, R₁₃, R₁₄, Y₂, [symbol] and [symbol] are as described herein for formula I or formula I′, to give the compound of formula I or formula I′ as described herein.

According to one embodiment, the method of the invention further comprises a step of reducing the compound of formula I or formula I′, using various methods known to those skilled in the art, to give the compound of formula I or formula I′, wherein Y₁ and Y₂ are identical and represent each CH₂ and wherein X₁, X₂, R₁, R₂, R₃, R₄, R₅, R₆, R₇, R₈, R₉, R₁₀, R₁₁, R₁₂, R₁₃, R₁₄, Y₁, Y₂, M, [symbol] and [symbol] are as described herein for formula I or formula I′.

According to another embodiment, the compound of formula X is synthesized using various methods known to those skilled in the art. According to one embodiment, the compound of formula X is synthesized in two steps by first reacting the pentose of formula XIV with the nitrogenous derivatives of formula XV, wherein R₁, R₁, R₂, R₃, R₄, R₅, R₆, R₇, Y₁ and R are as described herein for formula I or formula I′, to give the compound of formula X-1, then selectively deprotected to give the compound of formula X.

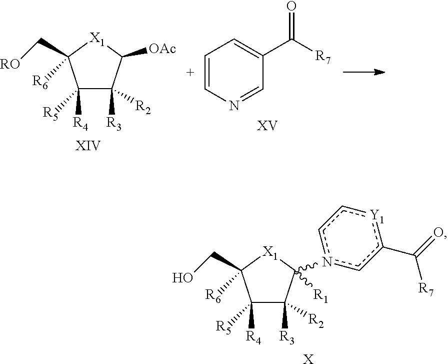

wherein X₁, R₁, R₂, R₃, R₄, R₅, R₆, R₇, Y₁, [symbol] and [symbol] are as described herein for formula I or formula I′ and R is a protecting group.

According to one embodiment, R is an appropriate protecting group known to those skilled in the art. Examples of appropriate protecting group include triarylmethyl and/or silyl groups. Non limiting examples of triarylmethyl includes trityl, monomethoxytrityl, 4,4′-dimethoxytrityl and 4,4′,4″-trimethoxytrityl. Non limiting examples of silyl groups includes trimethylsilyl, tert-butyldimethylsilyl, triisopropylsilyl, tert-butyldiphenylsilyl, tri-iso-propylsilyloxymethyl and [2-(trimethylsilyl)ethoxy]methyl.

According to one embodiment, any hydroxy group attached to the pentose ring is protected with an appropriate protecting group known to those skilled in the art.

The selection and exchange of the protecting groups is within the skill to those skilled in the art. Any protecting group can also be removed by methods known in the art, for example, with an acid (e.g., a mineral or an organic acid), a base or a fluoride source.

According to a preferred embodiment, the nitrogenous derivatives of formula XV is added to the pentose XIV via a coupling reaction in the presence of a Lewis acid to give the compound of formula X-1. Non limiting examples of suitable Lewis acid include TMSOTf, BF₃.OEt₂, TiCl₄ and FeCl₃.

According to a specific embodiment, the invention relates to a method for preparing the compound of formula VIII,

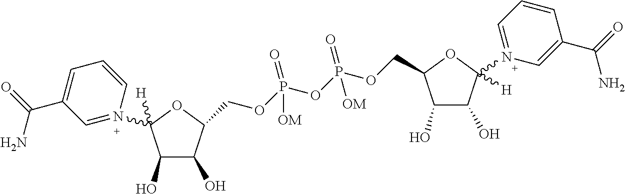

or pharmaceutically acceptable salts and/or solvates thereof.

In a first step, the nicotinamide of formula XV, is added to the ribose tetraacetate XIV, via a coupling reaction in the presence of a Lewis acid, to give the compound of formula X-1:

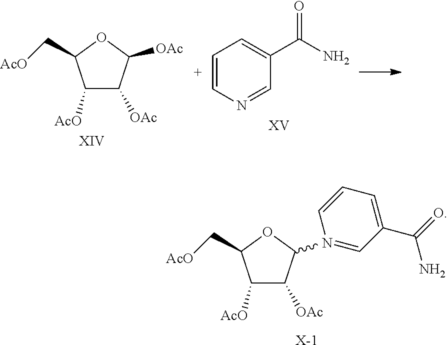

In a second step, an ammoniacal treatment of the compound of formula X-1 give the compound of formula X:

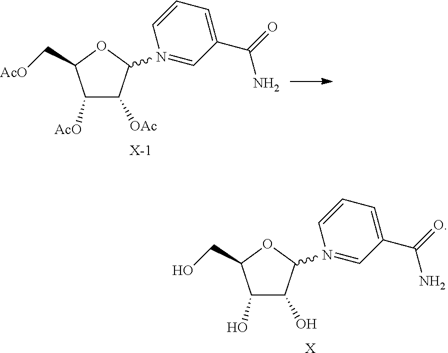

In a third step, the mono-phosphorylation of a compound of formula X, in the presence of phosphoryl chloride in a trialkyl phosphate, gives the phophorodichloridate compound XI:

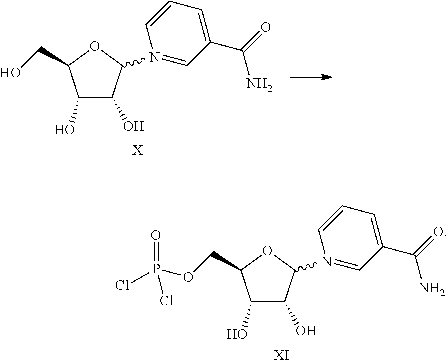

In a fourth step, the phophorodichloridate compound XI obtained in the third step is partially hydrolyzed to give the phosphate compound of formula XII:

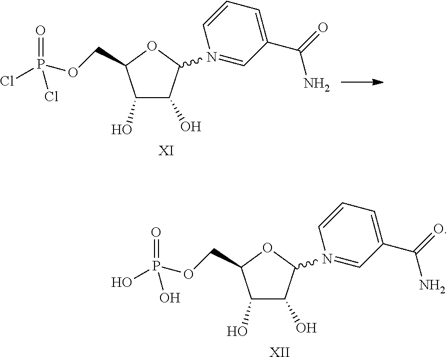

In a fifth step, the phosphate compound of formula XII obtained in the fourth step is then reacted, with the phophorodichloridate compound of formula XI obtained as described in the third step, to give the compound of formula VIII.

According to another specific embodiment, the invention relates to a method for preparing the compound of formula IX,

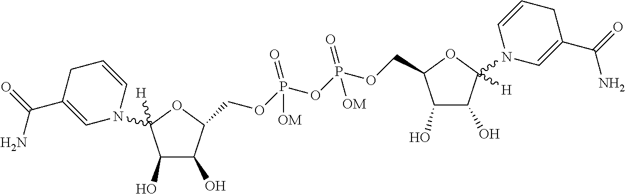

or pharmaceutically acceptable salts and/or solvates thereof.

According to one embodiment, the compound of formula IX is obtained from the compound of formula VIII, previously synthesized as described above.

In this embodiment, the compound of formula IX is obtained by reducing the compound of formula VIII, using a suitable reducing agent known to those skilled in the art, to give the compound of formula IX.

Use

The present invention further relates to the compound of formula I or formula I′ for use as a medicament.

According to one embodiment, the present invention relates to the compound of formula I or formula I′ for use in the treatment and/or prevention of a disease or disorder.

According to one embodiment, the disease or disorder is selected from age-related disorders, infectious diseases or parasitic diseases, neoplasms, cancers, diseases of the immune system, blood and organ diseases, endocrine, nutritional and metabolic diseases, mental, behavioural and neurodevelopmental disorders, sleep-wake disorders, diseases of the nervous system, diseases of the visual system, diseases of the ear and mastoid process, diseases of the circulatory system, vascular diseases, cardiomyopathies, diseases of the respiratory system, diseases of the digestive system, diseases of the skin and subcutaneous cell tissue, diseases of the musculoskeletal system or connective tissue, diseases of the genitourinary system, conditions related to sexual health, pregnancy, childbirth and puerperium, certain conditions originating in the perinatal period, developmental anomalies, congenital malformations and chromosomal anomalies, symptoms, signs and abnormal results of clinical and laboratory examinations, traumatic injuries, poisonings and some other consequences of external causes, external causes of morbidity and mortality, circadian clock modifications, kidney disorders, mitochondrial diseases, conditions or syndromes.

According to another embodiment, the present invention relates to the compound of formula I or formula I′ for use in the treatment of age-related disorders, infectious diseases or parasitic diseases, neoplasms, cancers, diseases of the immune system, blood and organ diseases, endocrine, nutritional and metabolic diseases, mental, behavioural and neurodevelopmental disorders, sleep-wake disorders, diseases of the nervous system, diseases of the visual system, diseases of the ear and mastoid process, diseases of the circulatory system, vascular diseases, cardiomyopathies, diseases of the respiratory system, diseases of the digestive system, diseases of the skin and subcutaneous cell tissue, diseases of the musculoskeletal system or connective tissue, diseases of the genitourinary system, conditions related to sexual health, pregnancy, childbirth and puerperium, certain conditions originating in the perinatal period, developmental anomalies, congenital malformations and chromosomal anomalies, symptoms, signs and abnormal results of clinical and laboratory examinations, traumatic injuries, poisonings and some other consequences of external causes, external causes of morbidity and mortality, circadian clock modifications, kidney disorders, mitochondrial diseases, conditions or syndromes.

According to one embodiment, the present invention relates to the compound of formula I or formula I′ for use in the treatment and/or prevention of age-related disorders.

Non-limiting examples of age-related disorders include lifespan, cellular senescence, progeria, frailty, osteoporosis, Werner's syndrome, muscle wasting.

According to one embodiment, the present invention relates to the compound of formula I or formula I′ as described herein above for use in the treatment and/or prevention of infectious diseases or parasitic diseases.

According to one embodiment, the infectious disease or parasitic disease is a gastroenteritis or colitis of infectious origin.

Non-limiting examples of gastroenteritis or colitis of infectious origin include:
Bacterial intestinal infections such as cholera, intestinal infection due to other Vibrio, intestinal infections due to Shigella, intestinal infections due to Escherichia coli, intestinal infections due to Clostridioides difficile, intestinal infections due to Yersinia enterocolitica, gastroenteritis due to Campylobacter, typhoid fever, paratyphoid fever, infections due to other Salmonella, other specified bacterial intestinal infections or unspecified bacterial intestinal infections;
Bacterial foodborne intoxications such as foodborne staphylococcal intoxication, botulism, foodborne Clostridium perfringens intoxication, foodborne Bacillus cereus intoxication, other specified bacterial foodborne intoxications or unspecified bacterial foodborne intoxications;
Viral intestinal infections such as enteritis due to Adenovirus, gastroenteritis due to Astrovirus, gastroenteritis due to Rotavirus, enteritis due to Norovirus, intestinal infections due to Cytomegalovirus, other specified viral intestinal infections or unspecified intestinal infections;
Protozoal intestinal infections such as infections due to Balantidium coli, giardiasis, cryptosporidiosis, cystoisosporiasis, sarcocystosis, blastocystosis, amoebiasis, other specified protozoal intestinal infections, unspecified protozoal intestinal infections or gastroenteritis or colitis without specification of infectious agent.

According to one embodiment, the infectious disease or parasitic disease is a predominantly sexually transmitted infection.

Non-limiting examples of predominantly sexually transmitted infections include:
Syphilis such as congenital syphilis, early syphilis, late syphilis, latent syphilis, unspecified as early or late;
Gonococcal infection such as gonococcal genitourinary infection, gonococcal pelviperitonitis, gonococcal infection of other sites, disseminated gonococcal infection or unspecified gonococcal infection;
Sexually transmissible infections due to chlamydia such as chlamydial lymphogranuloma, non-ulcerative sexually transmitted chlamydial infection, other specified sexually transmissible infections due to chlamydia, unspecified sexually transmissible infections due to chlamydia, chancroid, granuloma inguinale, trichomoniasis, sexually transmissible infestations, anogenital herpes simplex infection, anogenital warts, other specified predominantly sexually transmitted infections or predominantly sexually transmitted infections, unspecified.

According to one embodiment, the infectious disease or parasitic disease is a bacterial infection.

Non-limiting examples of bacterial infections include:
Bacterial diseases such as Actinomycosis, Bartonellosis, Whooping cough, Tetanus, Obstetrical tetanus, Tetanus neonatorum, Gas gangrene, Diphtheria, Brazilian purpuric fever, Legionellosis, Listeriosis, Nocardiosis, Meningococcal disease, Yaws, Pinta, Endemic non-venereal syphilis, Lyme borreliosis, Necrotising ulcerative gingivitis or Relapsing fever;
Mycobacterial disease such as tuberculosis of the respiratory system, tuberculosis of the nervous system, tuberculosis of other systems and organs, miliary tuberculosis, latent tuberculosis, leprosy, infections due to non-tuberculous mycobacteria, other specified mycobacterial diseases or unspecified mycobacterial diseases.
Staphylococcal or streptococcal diseases such as acute rheumatic fever without mention of heart involvement, acute rheumatic fever with heart involvement, rheumatic chorea, scarlet fever, streptococcal pharyngitis, toxic shock syndrome, meningitis due to Streptococcus, meningitis due to Staphylococcus, other specified staphylococcal or streptococcal diseases, staphylococcal or streptococcal diseases;
Zoonotic bacterial diseases such as rat-bite fevers, leptospirosis, glanders, plague, tularaemia, brucellosis, erysipeloid, anthrax, cat-scratch disease, pasteurellosis or extraintestinal yersiniosis;
Diseases due to chlamydiae such as chlamydial conjunctivitis, Chlamydial peritonitis, Infections due to Chlamydia psittaci or Trachoma;
Rickettsioses such as Typhus fever, potted fever, Rickettsialpox, Q fever, Campylobacteriosis, Melioidosis, Actinomycetoma, Non-pyogenic bacterial infections of the skin

According to one embodiment, the infectious disease or parasitic disease is a viral infection.

Non-limiting examples of viral infections include:
Viral diseases such as human immunodeficiency virus (HIV), Dengue virus, Mumps, Infectious mononucleosis, Cytomegaloviral disease, Epidemic myalgia, Viral conjunctivitis, Viral carditis, Viral haemorrhagic fever, Adenovirus infection, Enterovirus infection, Coronavirus infection, Parvovirus infection, influenza virus or viral hepatitis, adenovirus, human T-lymphotropic virus 1 (HTLV-1), Ebola virus;
Zoonotic viral diseases such as Filovirus disease, Arenavirus disease, Hantavirus disease, Henipavirus encephalitis, Middle East respiratory syndrome or Severe acute respiratory syndrome;
Arthropod-borne viral fever such as Chikungunya virus disease, Colorado tick fever, O'nyong-nyong fever, Oropouche virus disease, Rift Valley fever, Sandfly fever, West Nile virus infection, Yellow fever, Zika virus disease, Crimean-Congo haemorrhagic fever, Omsk haemorrhagic fever, Kyasanur Forest disease, Alkhurma haemorrhagic fever, Ross River disease or Severe fever with thrombocytopenia syndrome;
Infections due to poxvirus such as Smallpox, Monkeypox, Cowpox, Vaccinia, Buffalopox, Orf or Molluscum contagiosum;
Human papillomavirus infection of skin or mucous membrane such as common warts, Plane warts, Warts of lips or oral cavity or Wart virus proliferation in immune-deficient states;
Varicella zoster virus infections such as Varicella, Zoster, Herpes simplex infections, Roseola infantum, Rubella, Measles, Erythema infectiosum or Picornavirus infections presenting in the skin or mucous membranes.

According to one embodiment, the infectious disease or parasitic disease is a parasitic disease.

Non-limiting examples of parasitic diseases include:
Malaria such as malaria due to Plasmodium falciparum, malaria due to Plasmodium vivax, malaria due to Plasmodium malariae, malaria due to Plasmodium ovale, other parasitologically confirmed malaria or malaria without parasitological confirmation;
Nonintestinal protozoal diseases such as acanthamoebiasis, african trypanosomiasis, babesiosis, chagas disease, leishmaniasis, naegleriasis, rhinosporidiosis, toxoplasmosis or microsporidiosis;
Diseases due to nematodes such as angiostrongyliasis, anisakiasis, ascariasis, capillariasis, dracunculiasis, enterobiasis, filariasis, gnathostomiasis, hookworm diseases, oesophagostomiasis, onchocerciasis, strongyloidiasis, syngamosis, toxocariasis, trichinosis, trichostrongyliasis, trichuriasis or uncinariosis;
Diseases due to cestode such as cysticercosis, diphyllobothriasis, dipylidiasis, echinococcosis, hymenolepiasis, sparganosis or taeniasis
Diseases due to trematodes such as clonorchiasis, dicrocoeliasis, fascioliasis, fasciolopsiasis, opisthorchiasis, paragonimiasis, schistosomiasis, diphyllobothriasis and sparganosis or helminthiases;
Infestations by ectoparasites such as pediculosis, myiasis, external hirudiniasis, pthiriasis, scabies, tungiasis, cimicosis or infestation by mites.

According to one embodiment, the infectious disease or parasitic disease is a mycosis.

Non-limiting examples of mycoses include: aspergillosis, basidiobolomycosis, blastomycosis, candidosis, chromoblastomycosis, coccidioidomycosis, conidiobolomycosis, cryptococcosis, dermatophytosis, eumycetoma, histoplasmosis, lobomycosis, mucormycosis, non-dermatophyte superficial dermatomycoses, paracoccidioidomycosis, phaeohyphomycosis, pneumocystosis, scedosporiosis, sporotrichosis, talaromycosis or emmonsiosis.

According to one embodiment, the present invention relates to the compound of formula I or formula I′ as described herein above for use in the treatment or prevention of neoplasm.

Non-limiting examples of neoplasm include: neoplasms of brain or central nervous system, neoplasms of haematopoietic or lymphoid tissues, malignant neoplasms, malignant neoplasm of lip, oral cavity or pharynx, malignant neoplasm of digestive organs, malignant neoplasm of middle ear, respiratory or intrathoracic organs, malignant neoplasm of skin, malignant neoplasms of peripheral nerves or autonomic nervous system, malignant neoplasms of peripheral nerves or autonomic nervous system, malignant neoplasm of breast, malignant neoplasms of female genital organs, malignant neoplasms of male genital organs, malignant neoplasms of urinary tract, malignant neoplasms of eye or ocular adnexa, malignant neoplasms of endocrine glands, malignant neoplasm metastases, in situ neoplasm, benign neoplasm, benign mesenchymal neoplasms, benign non-mesenchymal neoplasms, benign neoplasm of respiratory or intrathoracic organs or benign cutaneous neoplasms.

According to another embodiment, the present invention relates to the compound of formula I or formula I′ as described herein above for use in the treatment or prevention of cancer.

Non-limiting examples of cancer include: cancer metastasis, brain cancer, kidney cancer, breast cancer, prostate cancer, testicular, ovarian, lymphomas, leukemias, pancreas, platinum-based chemotherapy induced ototoxicity, colon cancer, cancer of the large intestine, skin cancer, lung cancer, throat cancer, side effects of cancer chemotherapy.

According to another embodiment, the present invention relates to the compound of formula I or formula I′ as described herein above for use in the treatment or prevention of diseases of the immune system.

Non-limiting examples of diseases of the immune system include: primary immunodeficiencies, lupus, lupus erythematosus, systemic lupus erythematosus, idiopathic inflammatory myopathy, vasculitis, antiphospholipid syndrome, alopecia, ankylosing spondylitis, spondylarthritis, allergies, allergic reaction, scleroderma, Crohn's disease, anti-phospholipid antibody syndrome, Guillain-Barre syndrome, Lambert-Eaton syndrome, myasthenia gravis, Goodpasture syndrome or multiple sclerosis, disorders of neutrophil number, disorders of neutrophil function, eosinopenia, eosinophilia, disorders with decreased monocyte counts, disorders with increased monocyte counts, acquired lymphopenia, acquired lymphocytosis, sarcoidosis, polyclonal hypergammaglobulinaemia, cryoglobulinaemia, immune reconstitution inflammatory syndrome, graft-versus-host disease, diseases of thymus.

According to another embodiment, the present invention relates to the compound of formula I or formula I′ for use in the treatment or prevention of blood and organ diseases.

Non-limiting of examples of blood and organ diseases include: diseases related to platelet aggregation, blood clotting disorders, blood inflammation, materially inherited thrombocytopenia, materially inherited Leukemia syndrome, thrombotic disorders, thromboembolism, thrombophilia associated with anti-thrombin III deficiency, myeloproliferative disorders, disseminated intravascular coagulation, coagulation defects, thrombotic thrombocytopenia purpura, drug induced thrombocytopenia, dysfibrinogenemia, protein C deficiency, protein S deficiency, resistance to activated protein C, fibrinolytic disorders, sickle cell disease, sickle cell nutritional or metabolic anemias, haemolytic anemias, pure red cell aplasia, polycythaemia, sepsis, erythropoiesis or iron homeostasis.

According to another embodiment, the present invention relates to the compound of formula I or formula I′ and organ diseases for use in the treatment or prevention of endocrine, nutritional and metabolic diseases.

Non-limiting examples of endocrine diseases include:
Disorders of the thyroid gland or thyroid hormones system such as hypothyroidism, nontoxic goitre, thyrotoxicosis, thyroiditis, hypersecretion of calcitonin, generalized resistance to thyroid hormone or sick-euthyroid syndrome;
Diabetes mellitus such as type 1 diabetes mellitus, type 2 diabetes mellitus or malnutrition-related diabetes mellitus;
Disorders of glucose regulation or pancreatic internal secretion such as intermediate hyperglycaemia, hypoglycaemia without associated diabetes, increased secretion of glucagon, abnormal secretion of gastrin, insulin-resistance syndromes, persistent hyperinsulinaemic hypoglycaemia of infancy;
Disorders of the parathyroids or parathyroid hormone system such as hypoparathyroidism, hyperparathyroidism;
Disorders of the pituitary hormone system such as hyperfunction of pituitary gland, hypofunction;
Disorders of the adrenal glands or adrenal hormone system such as cushing syndrome, adrenogenital disorders, hyperaldosteronism, hypoaldosteronism, adrenocortical insufficiency or adrenomedullary hyperfunction;
Disorders of the gonadal hormone system such as ovarian dysfunction, testicular dysfunction or testosterone-related disorders,
Disorders of puberty such as disorder of puberty due to oestrogen resistance, delayed puberty or peripheral precocious puberty;
Polyglandular dysfunction such as autoimmune polyendocrinopathy or polyglandular hyperfunction.

Non-limiting examples of nutritional diseases include:
Undernutrition such as underweight in infants, children or adolescents, wasting in infants, children or adolescents, acute malnutrition in infants, children or adolescents stunting in infants, children or adolescents, underweight in adults, vitamin A deficiency, vitamin C deficiency, vitamin D deficiency, vitamin E deficiency, vitamin K deficiency, vitamin B1 deficiency, vitamin B2 deficiency, vitamin B3 deficiency, vitamin B6 deficiency, folate deficiency, vitamin B12 deficiency, biotin deficiency, pantothenic acid deficiency, choline deficiency or mineral deficiencies;
Sequelae of malnutrition;
Overweight, obesity or specific nutrient excesses such as overweight or obesity, overweight or localised adiposity, obesity, dietary obesity, hormone-related obesity, obesity-related to the administration of medication, obesity-related inflammation, vitamin excesses or mineral excesses.

Non-limiting examples of metabolic disorders include:
Inborn errors of metabolism such as inborn errors of amino acid or other organic acid metabolism, inborn errors of carbohydrate metabolism, inborn errors of lipid metabolism, inborn errors of energy metabolism, inborn errors of glycosylation or other specified protein modification, inborn errors of purine, pyrimidine or nucleotide metabolism, lysosomal diseases, peroxisomal diseases, inborn errors of porphyrin or heme metabolism, inborn errors of neurotransmitter metabolism, alpha-1-antitrypsin deficiency;
Disorders of metabolite absorption or transport such as disorders of amino acid absorption or transport, disorders of carbohydrate absorption or transport, disorders of lipid absorption or transport, disorders of vitamin or non-protein cofactor absorption or transport or disorders of mineral absorption or transport;
Disorders of fluid, electrolyte or acid-base balance such as volume depletion, hyperosmolality or hypernatraemia, hypo-osmolality or hyponatraemia, acidosis, alkalosis, mixed disorder of acid-base balance, hyperkalaemia, hypokalaemia or fluid overload;
Lipid disorders such as dyslipidemia/dyslipogenesis, hypercholesterolaemia, hyperlipidemia, Gaucher's disease, HDL hypocholesterolemia, LDL hypercholesterolemia, HLD non-cholesterolemia, hypertriglyceridemia, Niemann-Pick disease, Gangliosidosis (including Tay-Sachs disease), Leukodystrophies, Mucopolysaccharidose, Mucolipidose, Lipodystrophy, Beta-oxidation defect, HIV-induced lipodystrophy, Lipid storage myopathy.
Other metabolic disorders such as amyloidosis, tumour lysis syndrome, pre-diabetic state, diabetes insipidus, optic atrophy, deafness (DID-MOAD), diabetes mellitus and deafness (DMDF), impaired glucose tolerance, insulin resistance, diabetes related conditions or disorders, high blood glucose sugar level, high blood cholesterol, Hyperglycemia, homocystinuria, metabolic syndrome and syndrome X, glycoprotein storage disorder, Luft disease, CPT1 deficiency, CPT2 deficiency, hyperinsulinism.

According to another embodiment, the present invention relates to the compound of formula I or formula I′ as described herein above for use in the treatment or prevention of mental, behavioural and neurodevelopmental disorders.

Non-limiting examples of mental, behavioural and neurodevelopmental disorders include: schizophrenia, catatonia, behavioral and autism Spectrum disorders such as Asperger syndrome with declines during infection, autism with declines during infection, autism or Attention deficit hyperactivity disorder (ADHD); mood disorders such as bipolar disorders, depressive disorders, anxiety or fear-related disorders, obsessive-compulsive or related disorders, disorders specifically associated with stress, dissociative disorders, feeding or eating disorders, elimination disorders, disorders of bodily distress, distorted body image, low self-esteem; disorders due to substance use or addictive behaviours, impulse control disorders, disruptive behaviour or dissocial disorders; neurocognitive disorders such as dementia; mental or behavioural disorders associated with pregnancy, childbirth or puerperium.

According to another embodiment, the present invention relates to the compound of formula I or formula I′ as described herein above for use in the treatment or prevention of diseases of the nervous system.

Non-limiting examples of diseases of the nervous system include: inflammatory diseases of the central nervous system, such as bacterial meningitis, meningitis in bacterial diseases, meningitis in infectious diseases, encephalitis, myelitis, encephalomyelitis, intracranial and intraspinal abscess and granuloma, intracranial and intraspinal phlebitis and thrombophlebitis and sequelae of inflammatory diseases of central nervous system; systemic atrophies primarily affecting the central nervous system, such as Huntington's disease, hereditary ataxia, spinal muscular atrophy and related syndromes, systemic atrophies primarily affecting central nervous system and postpolio syndrome; extrapyramidal and movement disorders such as Parkinson disease, multiple system atrophy; other degenerative diseases of the nervous system such as Alzheimer's disease; Demyelinating diseases of the central nervous system such as multiple sclerosis, acute disseminated demyelination; Episodic and paroxysmal disorders such as epilepsy, status epilepticus, migraine, transient cerebral ischaemic attacks and related syndromes, vascular syndromes of brain in cerebrovascular diseases; Polyneuropathies and other disorders of the peripheral nervous system such as hereditary and idiopathic neuropathy, inflammatory polyneuropathy and Diseases of myoneural junction and muscle; essential tremor (ET), ataxia, catatonia, epilepsy, neuroleptic malignant syndrome, chorea, chorea gravidarum (chorea during pregnancy), chorea-acanthocytosis, cortical basal ganglional degeneration, dystonia, ischemic stroke, mental retardation, neuroacanthocytosis, Pelizaeus Merzbacher, X-linked spastic paraplegia, progressive supranuclear palsy (Steele-Richardson-Olszewski syndrome), Stratonigral degeneration, Shy-drager syndrome, Sporadic olivopontocerebellar, Creutzfeldt-Jakob disease, myelodisplasia, dementia, myoclonus and deafness (AMDF), Cockayne syndrome (Neill-Dingwall syndrome), Friedreich's ataxia, Alpers syndrome, Neuropathies, Chemotherapy-induced neuropathies, Diabetes-induced neuropathies, Amyotrophic lateral sclerosis (ALS), Primary lateral sclerosis, Lewy body dementia, Wolfram syndrome, Cerebral edema, Rett syndrome, Maternally inherited Leigh syndrome, Refractory epilepsy, Progressive Myoclonus Epilepsy (PME), Dyslexia with declines during infection, Cerebral palsy with declines during infection, Uridine responsive neurologic syndrome (URNS), Myoclonic Epilepsy and Psychomotor Regression (MERM), Familial Bilateral Striatal Necrosis (FBSN), Stroke, Striatonigral degeneration, Tardive dyskinesias, Charcot-Marie-Tooth disease, Post-traumatic head injury, Spinal cord injury, Familial migraines, Peripheral neuropathy, Tardy ulnar nerve palsy, Encephalomyelitis, Creatinine deficiency.

According to another embodiment, the present invention relates to a compound of formula I or formula I′ for use in the treatment and/or prevention of sleep-wake disorders.

Non-limiting examples of sleep-wake disorders include: insomnia disorders, hypersomnolence disorders, sleep-related breathing disorders, circadian rhythm sleep-wake disorders, sleep-related movement disorders, parasomnia disorders.

According to another embodiment, the present invention relates to a compound of formula I or formula I′ for use in the treatment and/or prevention of diseases of the visual system.

Non-limiting of examples of diseases of the visual system include: disorders of the ocular adnexa or orbit such as disorders of eyelid or peri-ocular area, disorders of lacrimal apparatus, disorders of orbit; disorders of the eyeball such as disorders of conjunctiva, disorders of the cornea, disorders of the anterior chamber, disorders of the anterior uvea, functional disorders of the pupil, disorders of lens, disorders of sclera, disorders of the choroid, disorders of the retina or disorders of the vitreous body; disorders of the visual pathways or centres; glaucoma or glaucoma suspect, retinal degeneration, age-related macular degeneration (AMD), retinitis pigmentosa (RP), rod and cone dystrophism, leber's congenital amaurosis (LCA), cataractocular disease (ocular neuritis), progressive external ophtalmoplegia, stargardt disease, hypertensive retinopathy, diabetic retinopathy, retinopathy, retinal haemorrhage, glaucoma, strabismus, esotropia, exotropia, hypermetropia, myopia, astigmatism, anisometropia, presbyopia, uveitis, conjunctivitis, photoreceptor degeneration following retinal detachments or optic neuropathy.

According to another embodiment, the present invention relates to a compound of formula I or formula I′ for use in the treatment and/or prevention of diseases of the ear and mastoid process.

Non-limiting of examples diseases of the ear and mastoid process include: infectious diseases of external ear, otitis externa such as noninfectious inflammation of external ear, noninflammatory disorders of the external ear; diseases of middle ear or mastoid such as otitis media, non-suppurative otitis media, suppurative otitis media; diseases of inner ear such as acute vestibular syndrome, episodic vestibular syndrome, chronic vestibular syndrome, otosclerosis, disorders of vestibular function, labyrinthine fistula, labyrinthine dysfunction, noise effects on inner ear; disorders with hearing impairment such as congenital hearing impairment such as acquired hearing impairment, deafness, Hearing loss, ototoxic hearing loss, Sensorineural Hearing Loss (SNHL), presbycusis, sudden idiopathic hearing loss, hereditary hearing loss, auditory synaptopathy or neuropathy; otalgia or effusion of ear, degenerative or vascular disorders of ear, disorders of acoustic nerve or ear atrophy.

According to another embodiment, the present invention relates to a compound of formula I or formula I′ for use in the treatment and/or prevention of diseases of the circulatory system.

Non-limiting of examples of diseases of the circulatory system include cardiac hypertrophy, Wolff-Parkinson-White syndrome, Atrial fibrillation, idiopathic ischemia/reperfusion, myocardial infarction, myocarditis, angina and unstable angina, cardiovascular diseases, hypertensive diseases, idiopathic hypotension, orthostatic hypotension, acute or chronic acute ischaemic heart diseases, diseases of coronary artery, pulmonary heart disease or diseases of pulmonary circulation such as pulmonary thromboembolism or pulmonary hypertension; acute, chronic or constrictive pericarditis, cardiac tamponade, haemopericardium, pericardial effusion; acute or subacute endocarditis; mitral valve diseases, aortic valve diseases, tricuspid valve diseases, pulmonary valve diseases; cardiac arrhythmia, heart failure such as congestive heart failure, congestive heart disease cardiac failure, left ventricular heart failure, high output syndromes, right heart failure, biventricular failure, diseases of arteries or arterioles, Brugada syndrome, heart failure with preserved ejection fraction; diseases of veins; disorders of lymphatic vessels or lymph nodes; postprocedural disorders of circulatory system; septic shock.

According to another embodiment, the present invention relates to a compound of formula I or formula I′ for use in the treatment and/or prevention of a vascular disease.

Non-limiting examples of vascular diseases include: atheromatous diseases, atheromatous of aorta, atheromatous of coronary arteries, atheromatous of carotid arteries, atheromatous of cerebrovascular arteries, atheromatous of renal arteries, atheromatous of iliac arteries, atheromatous of femoral arteries, atheromatous of popliteal arteries, retinal arterioles, glomerular arterioles, vasa nervorum, cardiac arterioles, capillary beds of the eye, the kidney, the heart and the central and peripheral nervous systems, restenosis (ex: following coronary intervention), disorders relating to an abnormal level of high and low density cholesterol, atherosclerosis, endothelial dysfunction, hypertension, high blood pressure, deep vein thrombosis, macrovascular diseases, perivascular diseases, vascular remodeling, giant cell arteritis, polyarteritis nodosa, vasculitis.

According to another embodiment, the present invention relates to the compound of formula I or formula I′ as described herein above for use in the treatment and/or prevention of cardiotoxicity.

Non-limiting examples of cardiotoxicity include: idiopathic cardiotoxicity, metabolic cardiotoxicity, alcoholic cardiotoxicity, drug-induced cardiotoxicity, ischemic cardiotoxicity, hypertensive cardiotoxicity, Maternally inherited Hypertrophic cardiotoxicity (MHCM), maternally inherited cardiotoxicity, maternal cardiotoxicity, fatal infantile cardiotoxicity Plus, MELAS-associated cardiotoxicity or Barth syndrome.

According to another embodiment, the present invention relates to a compound of formula I or formula I′ for use in the treatment and/or prevention of diseases of the respiratory system.

Non-limiting examples of diseases of the respiratory system include:
Upper respiratory tract disorders such as acute nasopharyngitis, acute sinusitis, acute pharyngitis, acute tonsillitis, acute laryngopharyngitis, acute laryngitis or tracheitis, acute obstructive laryngitis or epiglottitis, vasomotor or allergic rhinitis, chronic rhinitis, nasopharyngitis or pharyngitis, chronic rhinosinusitis, silent sinus syndrome, cyst or mucocele of nose or nasal sinus, deviated nasal septum, hypertrophy of nasal turbinates, chronic diseases of tonsils or adenoids, chronic laryngitis or laryngotracheitis, diseases of vocal cords or larynx, nasal polyp, abscess of upper respiratory tract;
Lower respiratory tract diseases such as bronchitis, emphysema, chronic obstructive pulmonary disease, asthma, bronchiectasis, cystic fibrosis, chronic bronchiolitis, tracheobronchitis;
Lung disorders such as pulmonary embolism, pulmonary hypertension, cystic fibrosis, asthma, respiratory infection, pulmonary infection, bronchitis, emphysema;
Lung infections such as pneumonia, post-influenza pneumonia, acute bronchiolitis, acute and chronic bronchitis, abscess of lung or mediastinum, pyothorax, idiopathic pulmonary fibrosis, acute lung injury, sarcoidosis;
Lung diseases due to external agents such as pneumoconiosis, pneumonitis hypersensitivity pneumonitis due to organic dust, pneumonitis due to solids and liquids, radiation pneumonitis Mendelson syndrome, airway disease due to specific organic dust, respiratory conditions due to inhalation of chemicals, gases, fumes or vapours, COPD (Chronic Obstructive Plumonary Disease);
Respiratory diseases principally affecting the lung interstitium such as acute respiratory distress syndrome, pulmonary oedema, pulmonary eosinophilia, idiopathic interstitial pneumonitis, primary interstitial lung diseases specific to infancy or childhood, interstitial lung diseases associated with systemic diseases, pulmonary alveolar microlithiasis, lymphangioleiomyomatosis;
Pleural, diaphragm or mediastinal disorders such as pleural plaque, pneumothorax, diseases of mediastinum, disorders of diaphragm, chylous effusion, fibrothorax, haemothorax, pleural effusion, respiratory failure;
Postprocedural disorders of the respiratory system such as tracheostomy malfunction, chronic pulmonary insufficiency following surgery, postprocedural subglottic stenosis, postprocedural stenosis of the trachea, transfusion related acute lung injury.

According to another embodiment, the present invention relates to a compound of formula I or formula I′ for use in the treatment and/or prevention of diseases of the digestive system.

Non-limiting examples of diseases of the digestive system include:
Diseases or disorders of orofacial complex such as disorders of lips, disorders of oral mucosa, diseases of tongue, diseases of salivary glands, cysts of oral or facial-neck region, certain specified diseases of jaws, disorders of tooth development or eruption, diseases of hard tissues of teeth, diseases of pulp or periapical tissues, gingival diseases, periodontal disease, sensory disturbances affecting orofacial complex;
Diseases of oesophagus such as acquired anatomical alterations of the oesophagus, motility disorders of oesophagus, gastro-oesophageal reflux disease, columnar metaplastic epithelium of the oesophagus, oesophagitis, oesophageal ulcer, vascular disorders of the oesophagus;
Diseases of stomach such as acquired anatomical alterations of the stomach, gastroduodenal motor or secretory disorders, gastritis, vascular disorders of the stomach, gastric polyp;
Diseases of duodenum such as acquired anatomical alterations of the duodenum, duodenitis, vascular disorders of the duodenum, duodenal polyp;
Intestinal disorders such as irritable Bowel syndrome, inflammatory Bowel disease, neurogenic Bowel dysfunction, chronic intestinal pseudo-obstruction with myopathy and ophtalmoplegia, Crohn's disease, colitis, ulcerative colitis, necrotizing enterocolitis, diarrhea, coeliac disease, motility disorders, diverticular diseases;
Hepatic diseases such as Non-alcoholic fatty liver disease (NAFLD), Non-alcoholic hepatic steatosis (NASH), hepatic fibrosis, hepatic cirrhosis, alcoholic liver disease, drug-induced or toxic liver diseases, autoimmune liver diseases, hepatitis steatosis, alcoholic hepatitis, infectious liver diseases, fulminant hepatitis, acute liver failure, hepatorenal syndrome, jaundice, hepatic vasculitis, cirrhosis, hemochromatosis, Wilson disease;
Diseases of gallbladder or biliary tract such as cholelithiasis, cholecystitis, cholangitis;
Diseases of pancreas such as cystic diseases of the pancreas, acute, chronic, autoimmune or obstructive pancreatitis;
Diseases of peritoneum such as peritonitis;
Ischemic vascular disorders of intestine such as acute vascular disorders of intestine or chronic vascular disorders of intestine.

According to another embodiment, the present invention relates to a compound of formula I or formula I′ for use in the treatment and/or prevention of diseases of the skin and subcutaneous cell tissue.

Non-limiting examples of skin diseases and subcutaneous cell tissue include: viral exanthems, dermatoses, dermatitis (including irritant contact dermatitis and allergic contact dermatitis), natural skin aging, wrinkles, liver spots/melasmas, skin inflammation, sun damage, atopic dermatitis, atopic eczema, seborrheic dermatitis, seborrheic keratosis, lichen simplex, asteatotic eczema, actinic keratosis, atrophy of the skin, keratinization disorders (including eczema), epidermolysis bullosa diseases (including penfigus), exfoliative dermatitis, seborrheic dermatitis, erythemas (erythema multiforme and erythema nodosum), damage caused by the sun or other light sources, discoid lupus erythematosus, dermatomyositis, lichen planus, lichen sclerosus, morphoea, psoriasis, lichenoid dermatoses, Pityriasis rubra pilaris, small plaque parapsoriasis, skin cancer, wound and burns (1st, 2nd and 3rd degree burns and/or thermal, chemical or electrical burns), graft rejections, xeroderma pigmentosum, hair loss, hair pigmentation, pemphigus, pemphigoid, linear IgA bullous dermatosis, epidermolysis bullosa acquisita, dermatitis herpetiformis acne, skin aging, urticaria, angioedema, cholinergic urticaria and related conditions, syndromes with urticarial reactions or angioedema, idiopathic angioedema, acute febrile neutrophilic dermatosis, pyoderma gangrenosum, erythema nodosum, genetic syndromes affecting the skin, epidermolysis bullosa simplex, junctional, dystrophic or syndromic epidermolysis bullosa, pruritus, prurigo, mucocutaneous or cutaneous pain syndromes, neuropathic skin damage, burning feet syndrome, ichthyoses, diffuse epidermal hyperkeratosis and acanthosis, porokeratosis, skin peeling, xerosis cutis or asteatosis, palmoplantar keratodermas, keratosis pilaris, acquired hypermelanosis, acquired melanotic macules or lentigines, endogenous non-melanin pigmentation, acquired hypomelanotic disorders, alopecia or hair loss, hypertrichosis, hirsutism and syndromes with hirsutism, acquired disorders of the hair shaft, acne, acneform inflammatory disorders, rosacea and related disorders, disorders of the sebaceous gland, hyperhidrosis, hypohidrosis, miliaria, acquired poikiloderma, keloid or hypertrophic scars, superficial fibromatoses, perforating dermatoses, necrobiotic granulomatous skin disorders, dermal dendrocyte, benign lymphocytic infiltration of the skin, lymphocytoma cutis, panniculitis, lipoatrophy or lipodystrophy, acquired malformations of cutaneous blood vessels, purpura or bruising due to disorders of coagulation, traumatic purpura, vasculitis or capillaritis involving the skin, livedoid vasculopathy, ichaemic ulceration of skin, lower limb venous eczema, vasodilatation of extremities, vasoconstriction of extremities, flushing disorders, skin disorders localised to the scalp, contact dermatitis of external ear, anal pruritus, infections of the anus or perianal skin, inflammatory dermatoses of the perianal area, sacrococcygeal pilonidal disease, neonatal viral infections involving the skin, neonatal pyogenic skin infections, neonatal fungal infections involving the skin, dermatoses of infancy, exanthematic drug eruption, drug-induced urticaria, angioedema and anaphylaxis, lichenoid drug eruption, Stevens-Johnson syndrome and toxic epidermal necrolysis due to drug, drug-induced erythroderma, dress syndrome, fixed drug eruption, acne or acneiform reactions attributable to drugs, drug eruption of other specified type, drug eruption of unspecified type, pigmentary abnormalities of skin due to drug, dermatoses precipitated by drug therapy, drug-induced hair abnormalities, drug-induced nail abnormalities, drug-induced oral conditions, photosensitivity due to drug, dermatoses associated with specific classes of medication, localised adverse cutaneous reactions to administration of drug, adverse cutaneous reactions to herbal, homoeopathic or other alternative therapies, pressure ulceration, dermatoses provoked by friction or mechanical stress, dermatoses due to foreign bodies, erythema ab igne, photoaging of the skin, allergic contact dermatitis, photo-allergic contact dermatitis, irritant contact dermatitis, allergic contact urticaria, protein contact dermatitis, allergic contact sensitisation, phototoxic dermatitis, cutaneous reactions to venomous or noxious animals, cutaneous cysts, skin tags or polyps, actinic keratosis and other discrete epidermal dysplasias, dermatoses which may presage cutaneous lymphoma, histiocytoses, paraneoplastic syndromes involving skin.

According to another embodiment, the present invention relates to a compound of formula I or formula I′ for use in the treatment and/or prevention of diseases of the musculoskeletal system or connective tissue.

Non-limiting examples of diseases of the musculoskeletal system or connective tissue include: osteoarthritis, infection related arthropathies, inflammatory arthropathies such as rheumatoid arthritis, psoriatic arthritis or polymyalgia rheumatica; spondylolysis, spinal stenosis, spondylolisthesis, inflammation of spine, spondylopathies, spondylarthritis, Synovitis acne pustulosis hyperostosis osteitis (SAPHO) syndrome, disorders of muscles, disorders of synovium or tendon, osteopathies, chondropathies, cachexia, muscular dystrophy, duchenne muscular dystrophy, becker muscular dystrophy, later-onset myopathy, congenital muscular dystrophy, maternal myopathy, myopathy, sarcopenia, bethlem myopathy, emery-dreifuss muscular dystrophy, facioscapulohumoral muscular dystrophy, hyaline body myopathy, limb-girdle myopathy, muscle sodium channel disorders, myotonic chondrodystrophy, myotubular myopathy, nemaline body disease, oculopharyngeal muscular dystrophy, spinal muscular atrophy, fibromyalgia, steroid myopathy, muscle weakness and atrophy, Gout, degenerative joint disease, joint inflammation, Marfan syndrome.

According to another embodiment, the present invention relates to a compound of formula I or formula I′ for use in the treatment and/or prevention of diseases of the genitourinary system.

Non-limiting examples of diseases of the genitourinary system include: disorders of the female genital tract such as vulvitis, inflammatory disorders of uterus, vaginitis, diseases of Bartholin gland, cervicitis, female pelvic inflammatory diseases, pelvic peritoneal adhesions, salpingitis and oophoritis, endometriosis, adenomyosis, dyspareunia, acquired abnormalities, abnormal uterine or vaginal bleeding such as menstrual irregularities; reproduction disorders such as miscarriage, eclampsia or pre-eclampsia, pregnancy, infertility, oopause infertility, male infertility, female infertility, irregular ovulation, chemotherapy-induced menopause; dermatoses of female genitalia; dermatoses of male genitalia; diseases of prostate such as hyperplasia of prostate, hydrocele, or spermatocele, orchitis or epididymitis; inflammatory disorders of male genital organs; vascular disorders of male genital system; disorders of breast such as benign breast disease, inflammatory disorders of breast, hypertrophy of breast; glomerular diseases such as nephritic syndrome, nephrotic syndrome, persistent proteinuria or albuminuria; renal tubulo-interstitial diseases such as acute tubulo-interstitial nephritis, acute pyelonephritis, acute tubular necrosis, acute renal papillary necrosis, tubulo-interstitial nephritis, chronic tubulo-interstitial nephritis, obstructive or reflux nephropathy, nephrocalcinosis, pyonephrosis, renal or perinephric abscess; kidney failure; urolithiasis; urethritis and urethral syndrome; urethral stricture; cystic or dysplastic kidney diseases; cystitis; gallstone; cholecystitis; cholelithiasis; neurogenic bladder dysfunction; postprocedural disorders of genitourinary system.

According to another embodiment, the present invention relates to a compound of formula I or formula I′ for use in the treatment and/or prevention of conditions related to sexual health.

Non-limiting examples of conditions related to sexual health include: sexual dysfunctions such as hypoactive sexual desire dysfunction, sexual arousal dysfunctions, orgasmic dysfunctions, ejaculatory dysfunctions; sexual pain disorders; or gender incongruence.

According to another embodiment, the present invention relates to a compound of formula I or formula I′ for use in the treatment and/or prevention of pregnancy, childbirth and puerperium.

Non-limiting examples of pregnancy, childbirth and puerperium include: abortive outcome of pregnancy; oedema, proteinuria, hypertensive disorders in pregnancy, childbirth or the puerperium; obstetric haemorrhage; maternal disorders predominantly related to pregnancy; maternal care related to the foetus, amniotic cavity or possible delivery problems; complications of labour or delivery; complications predominantly related to the puerperium.

According to another embodiment, the present invention relates to a compound of formula I or formula I′ for use in the treatment and/or prevention of certain conditions originating in the perinatal period.

Non-limiting examples of certain conditions originating in the perinatal period include: foetus or new-born affected by maternal factors or by complications of pregnancy, labour or delivery; disorders of new-born related to length of gestation or foetal growth; birth injury; infections of the foetus or new-born; haemorrhagic or haematological disorders of foetus or new-born; neurological disorders specific to the perinatal or neonatal period; respiratory disorders specific to the perinatal or neonatal period; cardiovascular disorders present in the perinatal or neonatal period; transitory endocrine or metabolic disorders specific to foetus or new-born; genitourinary system disorders specific to the perinatal or neonatal period; disorders involving the integument of foetus or new-born; skin disorders associated with prematurity; postnatal iatrogenic skin injury; disturbances of temperature regulation of new-born; certain disorders originating in the perinatal period. According to another embodiment, the present invention relates to a compound of formula I or formula I′ for use in the treatment and/or prevention of circadian clock modification.

Non-limiting examples of circadian clock modifications include: travel to or across one or more time zones, circadian clock, change in work shifts, night shift work, change in physical status (pregnancy or administration of medications), improper cycling or timing of feeding, fasting cycles, hyperglycemia, hypoglycemia, insomnia, advanced or delayed sleep phase syndrome, inconsistent sleep or wake cycles, narcolepsy or improve wakefulness in individuals suffering for excessive sleepiness.

According to another embodiment, the present invention relates to the compound of formula I or formula I′ as described herein above for use in the treatment and/or prevention of kidney disorders.

Non-limiting examples of kidney disorders include: kidney failure, renal ischemia/reperfusion injury (IRI), glomerular nephritis, lupus nephritis, nephropathy, glomerulosclerosis, nephrotic syndrome, hypertensive nephrosclerosis, acute nephritis, recurrent hematuria, persistent hematuria, chronic nephritis, rapidly progressive nephritis, acute renal failure, chronic renal failure, diabetic nephropathy, Bartter's syndrome, renal tubular acidosis.

According to another embodiment, the present invention relates to a compound of formula I or formula I′ for use in the treatment and/or prevention of mitochondrial diseases.

Non-limiting examples of mitochondrial diseases include: chronic progressive external ophtalmoplegia (KSS), myoclonus epilepsy associated with ragged-red fibers, Fukuhara syndrome (MERFF), mitochondrial Encephalopathy, lactic acidosis and stroke-like episodes (MELAS), Leber's hereditary optic neuropathy, leigh encephalopathia, Pearson's disease, Lactic acidosis, Lethal Infantile Mitochondriale Myopathy (LIMM), Mitochondrial encephalocardiomyopathy, Mitochondrial encephalomyopathy, mitochondrial myopathy, mitochondrial cytopathy, mitochondrial encephalopathy, multiple mitochondrial DNA deletion syndrome, mitochondrial DNA depletion syndrome, Pearson Marrow-Pancreas Syndrome, Multisystem mitochondrial disorder (myopathy, encephalopathy, blindness, hearing loss, peripheral neuropathy), Maternally inherited deafness or aminoglycoside-induced deafness, Myoneurogenic gastrointestinal encephalopathy, carnithine acylcarnithine transferase deficiency, carnithine deficiency, complex 1, 2, 3, 4, 5 deficiency.

According to another embodiment, the present invention relates to a compound of formula I or formula I′ for use in the treatment and/or prevention of a condition.

Non-limiting examples of conditions include: indigestion, fatigue, sleep disorders, canker sores, lethargy, energy deficiency, Pellagra, Gastrointestinal Reflux (GER), premenstrual syndrome, menstrual cramps, cyclic vomiting syndrome with declines during infection, exercise intolerance, stress urinary incontinence, chronic fatigue syndrome, stress, stress resistance, flushing, radiation or toxin-derived side effects, amino acids deficiency, transplantation, cell survival under hypoxia, co-enzyme Q10 deficiency, weight gain, obstructive sleep apnea, transplant rejection, endotoxin shock, exotoxin poisoning or chemotherapy fatigue.

According to another embodiment, the present invention relates to a compound of formula I or formula I′ for use in the treatment and/or prevention of a syndrome.

Non-limiting examples of syndromes include: epilepsy, Strokes, Optic atrophy and Cognitive Decline (ESOC), Myopathy and external ophtalmoplegia, Neuropathy, Gastro-intestinal, Encephalopathy (MNGIE), Neurogenic muscle weakness, neuropathy, ataxia and retinitis pigmentosa (NARP), Sudden Infant Death syndrome, Acquired immunodeficiency syndrome (AIDS), MARIAHS syndrome (Mitochondrial ataxia, recurrent infections, aphasia, hypouricemia/hypomyelination, seizures and dicarboxylic aciduria), Glutaric acid Type 2 deficiency/Long-Chain acyl-CoA dehydrogenase deficiency (DMAD), Autoimmune Polyglandular disease, Acute respiratory distress syndrome, Sjogren's syndrome, Granulocyte Transfusion Associated syndrome, Kearns-Sayre Syndrome, Reynaud syndrome.

According to a preferred embodiment, the present invention relates to the compound of formula I or formula I′ for use in the treatment of degenerative and neurodegenerative diseases.

Non-limiting examples of degenerative and neurodegenerative include: corticobasal syndrome, motor neuron diseases, systemic atrophies primarily affecting the central nervous system, Tay-Sachs disease, transmissible spongiform encephalopathies, ataxia-telangiectasia, autosomal dominant cerebellar ataxia, autosomal recessive spastic ataxia of Charlevoix-Saguenay, Baggio-Yoshinari syndrome, Batten disease, Cell-Cycle Hypothesis of Alzheimer's Disease, Cohen-Gibson syndrome, corticobasal degeneration, Creutzfeldt-Jakob disease, estrogen and neurodegenerative diseases, RAN translation, fatal insomnia, fragile X-associated tremor/ataxia syndrome, frontotemporal dementia and parkinsonism linked to chromosome 17, hereditary motor and sensory neuropathy with proximal dominance, infantile refsum disease, Kufor-Rakeb syndrome, Kufs disease, Locomotor ataxia, Lyme disease, Machado-Joseph disease, mental retardation and microcephaly with pontine and cerebellar hypoplasia, mitochondria associated membranes, multiple system atrophy, neuroacanthocytosis, Niemann-Pick disease, occupational exposure to Lyme disease, pontocerebellar hypoplasia, protein aggregation, pyruvate dehydrogenase deficiency, Sandhoff disease, spinocerebellar ataxia, subacute combined degeneration of spinal cord, subacute sclerosing panencephalitis, Tabes dorsalis, toxic encephalopathy, toxic leukoencephalopathy, transneuronal degeneration, Wobbly hedgehog syndrome.

According to a more preferred embodiment, the present invention relates to the compound of formula I or formula I′ for use in the treatment of pain, preferably nociceptive pain, even more preferably in the treatment of visceral pain.

According to one embodiment, the compound for use according to the invention or the compound according to the invention act on the nociception. According to one embodiment, the compound for use according to the invention or the compound according to the invention act on muscular pain. According to one embodiment, c the compound for use according to the invention or the compound according to the invention act on ligament pain. According to one embodiment, the compound for use according to the invention or the compound according to the invention act on tendon pain. According to one embodiment, the compound for use according to the invention or the compound according to the invention act on joint pain, such as knee pain, back pain, shoulder pain, neck pain, elbow pain, wrist pain, hip pain, ankle pain.

According to one embodiment, the pain is a symptom or a complication associated with a disease or disorder as described herein above.

According to one embodiment, the pain is a symptom or a complication associated with an antineoplastic-induced cardiotoxicity.

According to one embodiment, the pain is a symptom or a complication associated with sickle cell disease.

According to one embodiment, the present invention relates to the compound of formula I or formula I′ for use in the treatment of a drug-induced cardiotoxicity, more preferably in the treatment of an antineoplastic-induced cardiotoxicity.

According to one embodiment, the present invention relates to the compound of formula I or formula I′ for use in the treatment of sickle cell disease.

According to another embodiment, the present invention relates to the compound of formula I or formula I′ for use as a food supplement.

According to a preferred embodiment, the present invention relates to the compound of formula I or formula I′ for use as a food supplement to prevent cellular ageing by increasing NAD+ rate in the body.

According to another embodiment, the present invention relates to the compound of formula I or formula I′ for use as a cosmetic ingredient.

In another embodiment, the present invention relates to a food composition comprising at least one compound according to formula I or formula I′ or salts or solvates thereof, and at least one carrier and/or diluent.

In another embodiment, the present invention relates to a cosmetic composition comprising at least one compound according to formula I or formula I′ or salts or solvates thereof, and at least one carrier and/or diluent.

In another embodiment, the present invention relates to a pharmaceutical composition comprising at least one compound of the invention or pharmaceutically acceptable salts and/or solvates thereof, and at least one pharmaceutically acceptable carrier, diluent, excipient and/or adjuvant. The invention also covers a pharmaceutical composition which contains, in addition to at least one compound of the invention, or a pharmaceutically acceptable salt, solvate or pro-drug thereof as active ingredient, additional therapeutic agents and/or active ingredients.

The compound of the invention may be used in monotherapy or in combination therapy in a subject in need of therapeutic and/or preventive treatment. Thus, according to a first embodiment, the compound for use of the invention is administered to the subject without any other active ingredient. Thus, according to a second embodiment, the compound for use of the invention is administered to the subject in combination with at least one additional active ingredient, e.g., an active ingredient as described hereinabove.

In one embodiment, the compound is administrated to the subject sequentially, simultaneously and/or separately with the other active ingredient as described hereinabove.

Preferably, the subject in need of therapeutic and/or preventive treatment is a warm-blooded animal, more preferably a human. According to one embodiment, the subject is a male. According to one embodiment, the subject is a female.

According to one embodiment, the subject is an adult, i.e. over 18 years of age. According to one embodiment, the subject is a child, i.e. under 18 years of age. According to one embodiment, the subject is an infant, i.e. having an age of more than one month and less than two years. According to one embodiment, the subject is a newborn, i.e. having an age from birth to less than one month.

According to a preferred embodiment, the subject is of greater than 50, 55, 60, 65, 70, 75, 80, 85, 90 or 95 years of age. In one embodiment, the subject is of greater than 65, 70, 75, 80, 85, 90 or 95 years of age.

According to another preferred embodiment, the subject is of less than 20, 15, 10 or 5 years of age. In one embodiment, the subject is of less than 18, 17, 16, 15, 14, 13, 12, 11, 10, 9, 8, 7, 6, 5, 4, 3 or 2 years of age.

This invention also relates to the use of a compound or a pharmaceutical composition as described hereinabove as a medicament.

This invention also relates to the use of a compound or a pharmaceutical composition as described hereinabove for the treatment and/or prevention of a disease or disorder as described hereinabove.

This invention also relates to a method for the treatment and/or prevention of a disease or disorder as described hereinabove, comprising administering to a subject in need thereof of an effective amount of at least one compound or pharmaceutical composition as described hereinabove.

This invention also relates to the use of a compound or a pharmaceutical composition as described hereinabove in the manufacture of a medicament.

This invention also relates to the use of a compound or a pharmaceutical composition as described hereinabove in the manufacture of a medicament for the treatment and/or prevention of a disease or disorder as described hereinabove.

By means of non-limiting examples, such a formulation may be in a form suitable for oral administration, for parenteral administration (such as by intravenous, intramuscular or subcutaneous injection or intravenous infusion), for topical administration (including ocular), for administration by inhalation, by a skin patch, by an implant, by a suppository, etc. Such suitable administration forms—which may be solid, semi-solid or liquid, depending on the manner of administration—as well as methods and carriers, diluents and excipients for use in the preparation thereof, will be clear to the skilled person; reference is made to the latest edition of Remington's Pharmaceutical Sciences. Some preferred, but non-limiting examples of such preparations include tablets, pills, powders, lozenges, sachets, cachets, elixirs, suspensions, emulsions, solutions, syrups, aerosols, ointments, cremes, lotions, soft and hard gelatin capsules, suppositories, drops, sterile injectable solutions and sterile packaged powders (which are usually reconstituted prior to use) for administration as a bolus and/or for continuous administration, which may be formulated with carriers, excipients, and diluents that are suitable per se for such formulations, such as lactose, dextrose, sucrose, sorbitol, mannitol, starches, gum acacia, calcium phosphate, alginates, tragacanth, gelatin, calcium silicate, microcrystalline cellulose, polyvinylpyrrolidone, polyethylene glycol, cellulose, (sterile) water, methylcellulose, methyl- and propylhydroxybenzoates, talc, magnesium stearate, edible oils, vegetable oils and mineral oils or suitable mixtures thereof. The formulations can optionally contain other substances that are commonly used in pharmaceutical formulations, such as lubricating agents, wetting agents, emulsifying and suspending agents, dispersing agents, desintegrants, bulking agents, fillers, preserving agents, sweetening agents, flavoring agents, flow regulators, release agents, etc. The compositions may also be formulated so as to provide rapid, sustained or delayed release of the active compound(s) contained therein.

The pharmaceutical preparations of the invention are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use.

In another embodiment, the medicament of the invention comprises in addition to at least one compound of the invention, or a pharmaceutically acceptable salt, solvate or pro-drug thereof as active ingredients, additional therapeutic agents and/or active ingredients.

### BRIEF DESCRIPTION OF THE DRAWINGS

FIGS. 1A & C are graphs showing the basal nociceptive threshold for all experimental group.

FIGS. 1B & D are graphs showing the basal nociceptive scores for all experimental group.

FIG. 2A is a graph showing the nociceptive threshold for CYP-induced visceral pain at 2 h, 4 h and 6 h in comparison with basal value within the vehicle group. Friedman test or One-way Anova and Two-way RM Anova by both factors with Dunn's or Dunnett's post test vs Basal, ^{#}p<0.05, ^{##}p<0.01, ^{###}p<0.001, ^{####}p<0.0001.

FIG. 2B is a graph showing the nociceptive scores for CYP-induced visceral pain at 2 h, 4 h and 6 h in comparison with basal value within the vehicle group. Friedman test or One-way Anova and Two-way RM Anova by both factors with Dunn's or Dunnett's post test vs Basal, ^{#}p<0.05, ^{##}p<0.01, ^{###}p<0.001, ^{####}p<0.0001.

FIG. 3A is a graph showing the nociceptive threshold for the effects of NMN, pro-drug A and pro-drug B on CYP-induced allodynia at 2 h.

FIG. 3B is a graph showing the nociceptive threshold for the effects of NMN, pro-drug A and pro-drug B on CYP-induced allodynia at 4 h.

FIG. 3C is a graph showing the evolution of the nociceptive threshold following administration of compounds I-B and I-C over 6 hours. Two-way RM Anova with Sidak's multiple comparison test vs Vehicle group, ^{#}p<0.05, ^{##}p<0.01, ^{###}p<0.001, ^{####}p<0.0001.

FIG. 4 is a graph showing the effects NMN on CYP-induced visceral pain (nociceptive scores) at 2 h (FIG. 4A) and 4 h (FIG. 4B). Two-way RM Anova, ^{#}p<0.05, ^{##}p<0.01, ^{###}p<0.001, ^{####}p<0.0001

FIG. 5 is a graph showing the effects pro-drug A on CYP-induced visceral pain (nociceptive scores) at 2 h (FIG. 5A) and 4 h (FIG. 5B). Two-way RM Anova, ^{#}p<0.05, ^{##}p<0.01, ^{###}p<0.001, ^{####}p<0.0001.

FIG. 6 is a graph showing the effects pro-drug B on CYP-induced visceral pain (nociceptive scores) at 2 h (FIG. 6A) and 4 h (FIG. 6B). Two-way RM Anova, ^{#}p<0.05, ^{##}p<0.01, ^{###}p<0.001, ^{####}p<0.0001.

FIG. 7 is a graph showing the effects compound I-B on CYP-induced visceral pain (nociceptive scores). at 2 h (FIG. 7A), 4 h (FIG. 7B) and 6 h (FIG. 7C). Two-way RM Anova, ^{#}p<0.05, ^{##}p<0.01, ^{###}p<0.001, ^{####}p<0.0001.

FIG. 8 is a graph showing the effects compound I-C on CYP-induced visceral pain (nociceptive scores). at 2 h (FIG. 8A), 4 h (FIG. 8B) and 6 h (FIG. 8C). Two-way RM Anova, ^{#}p<0.05, ^{##}p<0.01, ^{###}p<0.001, ^{####}p<0.0001.

FIG. 9 is a histogram showing the survival rate of mice 5 days after DOX (20 mg/kg) or vehicle induction, with and without treatments. ##p<0.01: Fisher's test Dox mice treated with vehicle vs control mice, £p<0.05, ££p<0.01: Fisher's test Dox mice treated with vehicle vs Dox mice treated with NMN analogs.

FIG. 10A shows the body weight evolution of mice treated with NMN, compounds I-B and I-C (180 mg/kg) or vehicle, before (light gray symbol) and 5 days after saline solution or DOX (20 mg/kg) injection (dark gray symbol). £££p<0.001: Two-way ANOVA followed by Bonferroni post-test body weight before Dox injection vs 5 days after Dox injection.

FIG. 10B is a histogram showing the bodyweight gain calculated as follow: BW at the day of sacrifice minus BW before injection of mice treated with NMN, compounds I-B and I-C (180 mg/kg) or vehicle, with and without DOX (20 mg/kg) injection. ***p<0.001: Mann-Whitney test Dox mice treated with vehicle vs control mice, $$p<0.01, $$$p<0.001 One-way ANOVA followed by post-hoc Dunnett test Dox mice treated with vehicle n vs Dox mice treated with NMN analogs.

FIG. 11 is a histogram showing left ventricle (LV) end-diastolic (FIG. 11A) and end-systolic volumes (FIG. 11B), and ejection fraction (FIG. 11C) 5 days after saline solution or DOX (20 mg/kg) injection. **p<0.01, ***p<0.001: Mann-Whitney test Dox mice treated with vehicle vs control mice, $p<0.05, $$$p<0.001 Kruskal-Wallis test followed by post-hoc Dunn test Dox mice treated with vehicle vs Dox mice treated with NMN analogs.

FIG. 12 is a histogram showing LV end diastolic and end systolic diameters (FIGS. 12A and 12B respectively), fractional shortening (FIG. 12C) and heart rate (FIG. 12D) 5 days after saline solution or DOX (20 mg/kg) injection. **p<0.01, ***p<0.001: t-test or Mann-Whitney test Dox mice treated with vehicle vs control mice, $$$p<0.001: One-way ANOVA followed by post-hoc Dunnett test or Kruskal-Wallis test followed by post-hoc Dunn Dox mice treated with vehicle vs Dox mice treated with NMN analogs (180 mg/kg) or vehicle.

FIG. 13 is a histogram showing LV anterior wall thickness in systole and in diastole (FIGS. 13A and 13B respectively) and posterior wall thickness in systole and in diastole (FIGS. 13C and 13D respectively) 5 days after saline solution or DOX (20 mg/kg) injection. *p<0.05, **p<0.01: Mann-Whitney test Dox mice treated with vehicle vs control mice.

FIG. 14 is a histogram graph showing heart weight (FIG. 14A) and heart weight normalized to tibial length (FIG. 14B) 5 days after saline solution or DOX (20 mg/kg) injection. ***p<0.001: t-test Dox mice treated with vehicle vs control mice.

FIG. 15 is a histogram graph showing LDH concentrations (U/L, FIG. 15A) and LDH (fold change, FIG. 15B) in the plasma of mice 5 days after saline solution or DOX (20 mg/kg) injection. **p<0.01: Mann-Whitney test Dox mice treated with vehicle vs control mice; $p<0.05: Kruskal-Wallis test Dox mice treated with vehicle vs Dox mice treated with NMN analogs (180 mg/kg) or vehicle.

FIG. 16 is a histogram graph showing the ability of NMN to prevent sickling of SS RBCs at a 1% O₂. Non-parametric one-way ANOVA followed by Kruskal-Wallis test: *p<0.05; **p<0.01; ***p<0.001; ****p<0.0001.

FIG. 17 is a histogram graph showing the ability of compounds I-B to prevent sickling of SS RBCs at a 1% O₂. Non-parametric one-way ANOVA followed by Kruskal-Wallis test: *p<0.05; **p<0.01; ***p<0.001; ****p<0.0001.

FIG. 18 is a histogram graph showing the ability of compounds I-C to prevent sickling of SS RBCs at a 1% O₂. Non-parametric one-way ANOVA followed by Kruskal-Wallis test: *p<0.05; **p<0.01; ***p<0.001; ****p<0.0001.

### EXAMPLES

The present invention is further illustrated by the following examples.

### Example 1: Synthesis of Compounds of the Invention

### Material and Methods

All materials were obtained from commercial suppliers and used without further purification. Thin-layer chromatography was performed on TLC plastic sheets of silica gel 60F254 (layer thickness 0.2 mm) from Merck. Column chromatography purification was carried out on silica gel 60 (70-230 mesh ASTM, Merck). Melting points were determined either on a digital melting point apparatus (Electrothermal IA 8103) and are uncorrected or on a Kofler bench type WME (Wagner & Munz). IR, ¹H, ¹⁹F and ¹³C NMR spectra confirmed the structures of all compounds. IR spectra were recorded on a Perkin Elmer Spectrum 100 FT-IR spectrometer and NMR spectra were recorded, using CDCl₃, CD₃CN, D₂O or DMSO-d₆ as solvent, on a BRUKER AC 300 or 400 spectrometer at 300 or 400 MHz for ¹H, 75 or 100 MHz for ¹³C and 282 or 377 MHz for ¹⁹F spectra. Chemical shifts (δ) were expressed in parts per million relative to the signal indirectly (i) to CHCl₃ (δ 7.27) for ¹H and (ii) to CDCl₃ (δ 77.2) for ¹³C and directly (iii) to CFCl₃ (internal standard) (δ 0) for ¹⁹F. Chemical shifts are given in ppm and peak multiplicities are designated as follows: s, singlet; br s, broad singlet; d, doublet; dd, doublet of doublet; t, triplet; q, quadruplet; quint, quintuplet; m, multiplet. High resolution mass spectra (HRMS) were obtained from the “Service Central d'analyse de Solaize” (Centre Nationale de la Recherche Scientifique) and were recorded on a Waters spectrometer using electrospray ionization-TOF (ESI-TOF).

### General Experimental Procedures

### Step 1: Synthesis of Compound of Formula X-1

Compound of formula XIV (1.0 equiv.) is dissolved in dichloromethane. Nicotinamide of formula XV (1.50 equiv.) and TMSOTf (1.55 equiv.) are added at room temperature. The reaction mixture is heated to reflux and stirred until completion is reached. The mixture is cooled down to room temperature and filtered. The filtrate is concentrated to dryness to give the crude NR tetraacetate of formula X-1.

### Step 2: Synthesis of Compound of Formula X

The crude NR tetraacetate of formula X-1 is dissolved in methanol and cooled down to −10° C. 4.6 M Ammonia in methanol (3.0 equiv.) is added at −10° C. and the mixture is stirred at this temperature until completion is reached. Dowex HCR (H+) is added until pH=6-7. The reaction mixture is warmed to 0° C. and filtered. The resin is washed with a mixture of methanol and acetonitrile. The filtrate is concentrated to dryness. The residue is dissolved in acetonitrile and concentrated to dryness. The residue is dissolved in acetonitrile to give a solution of crude NR triflate of formula X.

### Step 3: Synthesis of Compound of Formula XI

The solution of crude NR triflate in acetonitrile is diluted with trimethyl phosphate (10.0 equiv.). Acetonitrile is distilled under vacuum and the mixture is cooled to −10° C. Phosphorus oxychloride (4.0 equiv.) is added at −10° C. and the mixture is stirred at −10° C. until completion.

### Step 4 and Step 5: Synthesis of Compound of Formula I-A

The mixture is hydrolyzed by addition of a 50/50 mixture of acetonitrile and water, followed by addition of tert-butyl methyl ether. The mixture is filtered and the solid is dissolved in water. The aqueous solution is neutralized by addition of sodium bicarbonate and extracted with dichloromethane. The aqueous layer is concentrated to dryness to give a crude mixture of NMN and di-NMN of formula I-A.

### Isolation of Di-NMN of Formula I-A:

NMN and di-NMN of formula I-A are separated by purification on Dowex 50wx8 with water elution. The fractions containing di-NMN are concentrated to dryness. The residue is purified by column chromatography on silica gel (gradient isopropanol/water). Pure fractions are combined and concentrated. The residue is freeze-dried to afford di-NMN as a beige solid.

³¹P RMN: δ (ppm, reference 85% H₃PO₄: 0 ppm dans D₂O)=−11.72; ¹H RMN: δ (ppm, reference TMS: 0 ppm dans D₂O)=4.20 (ddd, J_{H-H}=11.9, 3.5, 2.4 Hz, 2H), 4.35 (ddd, J_{H-H}=11.9, 3.9, 2.2 Hz, 2H), 4.43 (dd, J_{H-H}=5.0, 2.6 Hz, 2H), 4.53 (t, J_{H-H}=5.0 Hz, 2H), 4.59 (m, 2H), 6.16 (d, J_{H-H}=5.4 Hz, 2H), 8.26 (dd, J_{H-H}=8.1, 6.3 Hz, 2H), 8.93 (d, J_{H-H}=8.1 Hz, 2H), 9.25 (d, J_{H-H}=6.2 Hz, 2H), 9.41 (s, 2H); ¹³C RMN: δ (ppm, reference TMS: 0 ppm dans D₂O)=64.84 (CH₂), 70.73 (CH), 77.52 (CH), 87.11 (CH), 99.88 (CH), 128.65 (CH), 133.89 (Cq), 139.84 (CH), 142.54 (CH), 146.04 (CH), 165.64 (Cq); MS (ES+): m/z=122.8 [Mnicotinamide+H]+, 650.8 [M+H]+.

Synthesis of Compound of Formula I-B Phosphorus oxychloride (3.0 eq.) is added to trimethylphosphate (20.0 eq.) at −5° C. β-NR chloride (1.0 eq.) is added by portions at −5° C. and the reaction mixture stirred overnight at −5° C. Morpholine (3.0 eq.) is added dropwise at −10/0° C. and the mixture stirred for 2-3 h. α-NMN (1.0 eq.) is then added by portions at −5° C. and the reaction mixture stirred at −5° C. overnight. Hydrolysis is performed by dropwise addition of water (5 vol.) at −10/0° C. and the mixture is stirred until complete homogeneization at 10-15° C. The reaction mixture is then extracted with dichloromethane (6*10 vol.) and the aqueous phase neutralized by eluting through Purolite A600E formate form resin (theoretical amount to neutralize HCl coming from POCl₃). The eluate is then concentrated on vacuum at 45/50° C. to give the crude containing the α,β-diNMN of formula I-B. Elution with water through Dowex 50wx8 100-200 mesh H⁺ form resin allows removing of some impurities. Fractions containing compound I-B are combined and concentrated on vacuum at 45-50° C. The crude is then purified by preparative chromatography on Luna Polar RP 10 μm stationary phase with elution with a 10 mM NaH₂PO₄ aqueous solution. Pure fractions are combined and eluted with water on Purolite C100EH H⁺ form resin (needed quantity to fully exchange Na⁺ by H⁺), then eluted on Purolite A600E acetate form resin (needed quantity to fully exchange H₂PO₄⁻ by acetate). The eluate is concentrated on vacuum and the residue freeze-dried to afford compound I-B as a white solid.

³¹P RMN: δ (ppm, reference 85% H₃PO₄: 0 ppm dans D₂O)=−11.87, −11.69, −11.46, −11.29; ¹H RMN: δ (ppm, reference TMS: 0 ppm dans D₂O)=4.10 (ddd, J=11.1, 6.1, 3.1 Hz, 1H), 4.15-4.25 (m, 2H), 4.36 (ddd, J=12.2, 4.4, 2.4 Hz, 1H), 4.40 (dd, J=4.9, 2.4 Hz, 1H), 4.44 (dd, J=5.0, 2.7 Hz, 1H), 4.53 (t, J=5.0 Hz, 1H), 4.5 (m, 1H), 4.85 (m, 1H), 4.92 (t, J=5.3 Hz, 1H), 6.15 (d, J=5.5 Hz, 1H), 6.51 (d, J=5.7 Hz, 1H), 8.14 (dd, J=8.0, 6.3 Hz, 1H), 8.26 (dd, J=8.1, 6.3 Hz, 1H), 8.88 (d, J=8.1 Hz, 1H), 8.92 (d, J=8.1 Hz, 1H), 9.02 (d, J=6.3 Hz, 1H), 9.24 (s, 1H), 9.26 (d, J=6.4 Hz, 1H), 9.40 (s, 1H); ¹³C RMN: δ (ppm, reference TMS: 0 ppm dans D₂O)=64.83, 64.87 (CH2), 65.30, 65.35 (CH2), 70.65 (CH), 70.74 (CH), 71.92 (CH), 77.51 (CH), 87.03, 87.10 (CH), 87.19, 87.26 (CH), 96.57 (CH), 99.83 (CH), 126.89 (CH), 128.54 (CH), 132.44 (Cq), 133.81 (Cq), 139.85 (CH), 140.92 (CH), 142.50 (CH), 143.49 (CH), 145.06 (CH), 145.97 (CH), 165.64 (Cq), 165.88 (Cq); MS (ES+): m/z=122.8 [Mnicotinamide+H]+, 650.9 [M+H]+.

### Synthesis of Compound of Formula I-C

Phosphorus oxychloride (3.0 eq.) is added to trimethylphosphate (20.0 eq.) at −5° C. α-NR chloride (1.0 eq.) is added by portions at −5° C. and the reaction mixture stirred overnight at −5° C. Morpholine (3.0 eq.) is added dropwise at −10/0° C. and the mixture stirred for 2-3 h. α-NMN (1.0 eq.) is then added by portions at −5° C. and the reaction mixture stirred at −5° C. overnight. Hydrolysis is performed by dropwise addition of water (5 vol.) at −10/0° C. and the mixture is stirred until complete homogenization at 10-15° C. The reaction mixture is then extracted with dichloromethane (6*10 vol.) and the aqueous phase neutralized by eluting through Purolite A600E formate form resin (theoretical amount to neutralize HCl coming from POCl₃). The eluate is then concentrated on vacuum at 45/50° C. to give the crude containing the α,α-diNMN of formula I-C. Elution with water through Dowex 50wx8 100-200 mesh H⁺ form resin allows removing of some impurities. Fractions containing the compound I-C are combined and concentrated on vacuum at 45-50° C. The crude is then purified by preparative chromatography on Luna Polar RP 10 μm stationary phase with elution with a 10 mM NaH₂PO₄ aqueous solution. Pure fractions are combined and eluted with water on Purolite C100EH H⁺ form resin (needed quantity to fully exchange Na⁺by H⁺), then eluted on Purolite A600E acetate form resin (needed quantity to fully exchange H₂PO₄⁻ by acetate). The eluate is concentrated on vacuum and the residue freeze-dried to afford compound I-C as a white solid.

³¹P RMN: δ (ppm, reference 85% H₃PO₄: 0 ppm dans D₂O)=−11.40; ¹H RMN: δ (ppm, reference TMS: 0 ppm dans D₂O)=4.14 (ddd, J=11.4, 3.4, 2.8 Hz, 2H), 4.23 (ddd, J=11.6, 3.3, 2.8 Hz, 2H), 4.44 (dd, J=4.8, 2.3 Hz, 2H), 4.88 (m, 2H), 4.96 (t, J=5.3 Hz, 2H), 6.54 (d, J=5.7 Hz, 2H), 8.15 (dd, J=8.1, 6.2 Hz, 2H), 8.89 (d, J=8.1 Hz, 2H), 9.05 (d, J=6.3 Hz, 2H), 9.26 (s, 2H); ¹³C RMN: δ (ppm, reference TMS: 0 ppm dans D₂O)=65.37 (CH2), 70.70 (CH), 71.95 (CH), 87.30 (CH), 96.62 (CH), 126.91 (CH), 132.45 (Cq), 140.94 (CH), 143.52 (CH), 145.07 (CH), 165.90 (Cq); MS (ES+): m/z=122.7[Mnicotinamide+H]+, 650.8 [M+H]+.

### Example 2: Evaluation of Compounds of the Invention in the Acute Cyclophosphamide (CYP)-Induced Cystitis Model in Female Sprague-Dawley Rats

The aim of the present study was to evaluate, the effects of oral administration of Nicotinamide Mono-Nucleoside (NMN), Pro-drug A (alpha-NMN) and Pro-drug B (compound I-A), compounds I-B and I-C at 500 mg/kg on visceral pain response in the acute cyclophosphamide (CYP)-induced cystitis model in female Sprague-Dawley rats.

### I. Materials and Methods

### Animals

Female Sprague-Dawley rats, 7 weeks at delivery

### Pharmacological Treatment

NMN: 500 mg/kg
Pro-drug A: 500 mg/kg
Pro-drug B (compound I-A): 500 mg/kg
Compound I-B: 500 mg/kg
Compound I-C; 500 mg/kg
Vehicle: distilled water
Route of administration: per os (p.o.), 5 mL/kg
Frequency of administration: once at DO, 15 min prior CYP intraperitoneal (i.p.) injection.

### CYP-Induced Acute Cystitis

CYP was injected i.p. at 150 mg/kg in a final volume of 5 mL/kg in saline.

### Mechanical Stimulation Using Von Frey Filaments

Rats were placed in individual Plexiglas boxes with a wire mesh floor and allowed to adapt to the chamber for at least 30 min before any test starts.
8 von Frey filaments with increasing forces of 1, 2, 4, 6, 8, 10, 15 and 26 g were used.
Each calibrated filament was applied 3 times in the lower abdominal area close to the urinary bladder.

### Nociceptive Behaviors Scoring for Each Application

Score 0=no response
Score 1=retraction of the abdomen
Score 2=trampling or change of position
Score 3=flinching or abdominal curvature or licking of the site stimulated with von Frey filaments
   For Each Rat, Results were Expressed as:
Nociceptive threshold: first von Frey force for which the stimulus is perceived as painful (score ≥1 is obtained)

=>lowered threshold=allodynia
Nociceptive score: % of the maximal response (total=9 for 3 pooled applications) for each filament

=>Global pain response

### Experimental Group:

TABLE 2

| Group | Treatment | Injection | n |
|---|---|---|---|
| 1 | Vehicle (5 mL/kg) | CYP | 6 |
| 2 | NMN (500 mg/kg) | CYP | 6 |
| 3 | Pro-drug A (500 mg/kg) | CYP | 6 |
| 4 | Pro-drug B (500 mg/kg) | CYP | 6 |
| 5 | Compound I-B (500 mg/kg) | CYP | 6 |
| 6 | Compound I-C (500 mg/kg) | CYP | 6 |

### II. Results and Discussion

1. Basal Nociceptive Parameters (Before CYP Injection) for all Experimental Groups

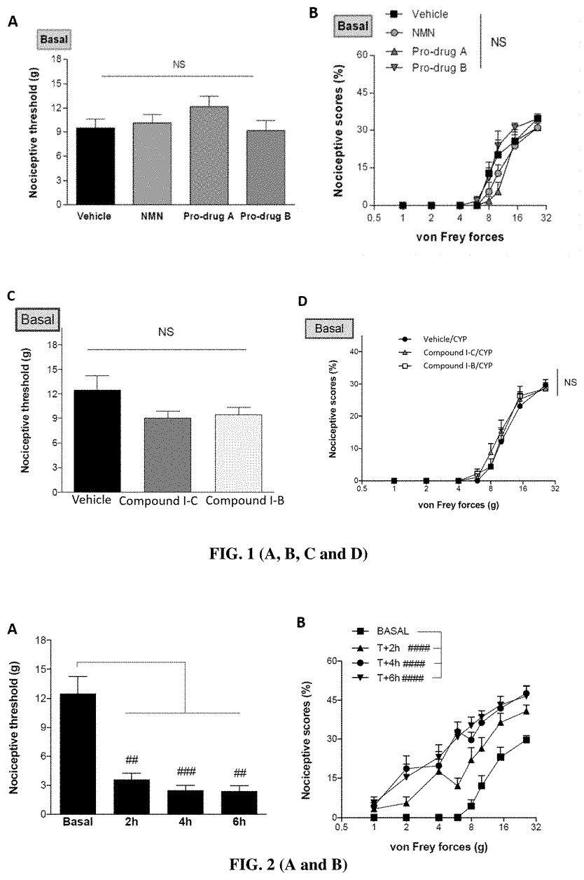

The results show (FIGS. 1A, 1B, 1C and 1D) that basal nociceptive responses were similar between all experimental groups (before CYP injection).

2. CYP-Induced Visceral Pain at 2 h and 4 h Post-Injection (in Comparison with Basal Value within the Vehicle Group)

The results show that in comparison to basal response, CYP (150 mg/kg, i.p.) induced a significant decrease in nociceptive threshold (FIG. 2A) and a significant increase in nociceptive scores (FIG. 2B) at 2 h, 4 h and 6 h.

3. Effect of NMN, Pro-Drug a, Pro-Drug B, Compound I-B and Compound I-C on CYP-Induced Allodynia (Nociceptive Threshold)

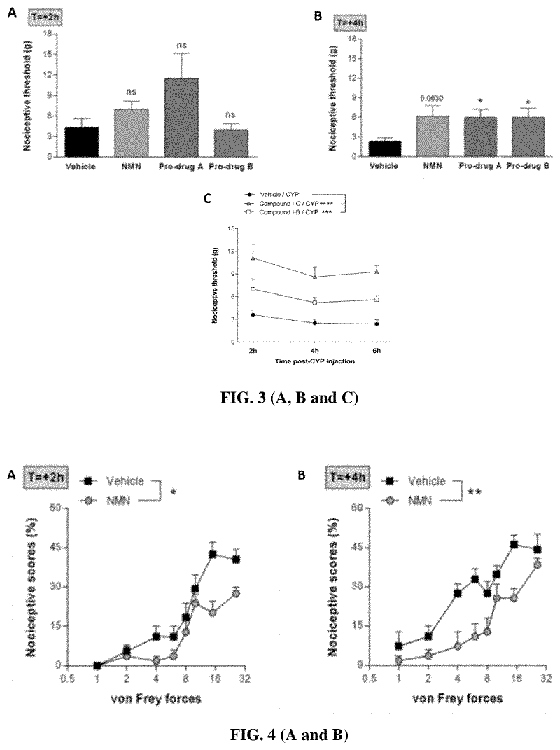

The results show that in comparison to vehicle:
NMN (500 mg/kg, p.o) led to a slight increase in nociceptive threshold at +2 h (FIG. 3A) and +4 h (FIG. 3B) with an effect just above the margin of statistical significance at +4 h (p=0.063),
Pro-Drug A (500 mg/kg, p.o) led to a non-significant increase in nociceptive threshold at +2 h (FIG. 3A) and a significant increase at +4 h (FIG. 3B),
Pro-Drug B (500 mg/kg, p.o) led to a significant increase in nociceptive threshold at +4 h (FIG. 3B).
Compound I-B (500 mg/kg, p.o) led to a significant increase in nociceptive threshold at +6 h following CYP induction (FIG. 3C).
Compound I-C (500 mg/kg, p.o) led to a significant increase in nociceptive threshold at +6 h following CYP induction (FIG. 3C).

4. Effects of NMN, Pro-Drug A and Pro-Drug B, Compounds I-B and I-C on CYP-Induced Visceral Pain (Nociceptive Scores)

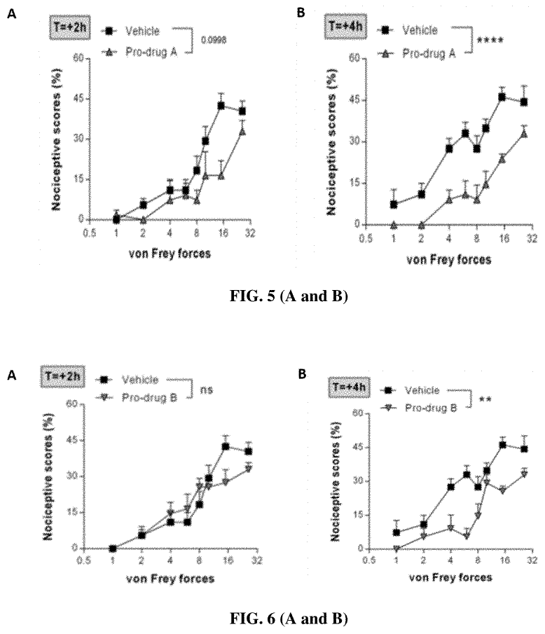

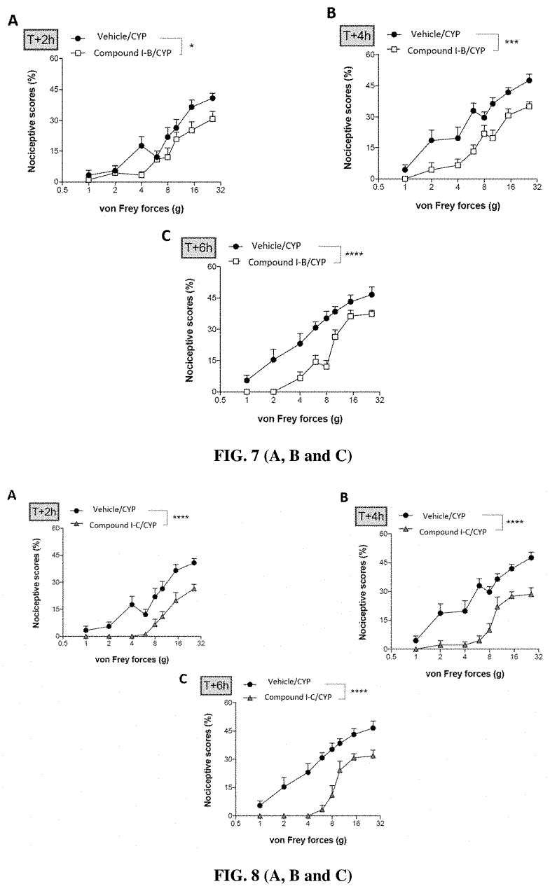

The results show that in comparison to vehicle:
NMN (500 mg/kg, p.o) led to a significant decrease in nociceptive scores at +2 (FIG. 4A) and +4 h (FIG. 4B),
Pro-Drug A (500 mg/kg, p.o) led to a decrease in nociceptive scores at +2 (FIG. 5A) and +4 h (FIG. 5B) that achieved the statistical level only at +4 h,
Pro-Drug B (500 mg/kg, p.o) led to a significant decrease in nociceptive scores at +4 h (FIG. 6B) (no effect was observed at +2 h (FIG. 6A)),
Compound I-B (500 mg/kg, p.o) led to a significant decrease in nociceptive scores at +2 (FIG. 7A), +4 h (FIG. 7B) and +6 h (FIG. 7C),
Compound I-C (500 mg/kg, p.o) led to a significant decrease in nociceptive scores at +2 (FIG. 8A), +4 h (FIG. 8B) and +6 h (FIG. 8C).

5. Summary of Results

Basal nociceptive responses were similar between all experimental groups (before CYP injection).

In comparison to basal response, effects of CYP (150 mg/kg, i.p.) at 2 and 4 hours were characterized by:
A significant decrease in nociceptive threshold at +2, +4 h and +6 h,
A significant increase in nociceptive scores at +2, +4 h and +6 h.

In comparison to vehicle, in CYP-injected rats, effects of NMN (500 mg/kg, p.o.) led to:
A slight increase in nociceptive threshold at +2 and +4 h with an effect just above the margin of statistical significance at +4 h (p=0.063),
A significant decrease in nociceptive scores at +2 and +4 h.

In comparison to vehicle, in CYP-injected rats, effects of Pro-drug A (500 mg/kg, p.o.) were characterized by:
An increase in nociceptive threshold at +2 and +4 h with an effect that reached significance at +4 h,
A decrease in nociceptive scores at +2 and +4 h that achieved significance at +4 h.

In comparison to vehicle, in CYP-injected rats, effects of Pro-drug B (500 mg/kg, p.o.) led to:
A significant increase in nociceptive threshold at +4 h,
A significant decrease in nociceptive scores at +4 h.

In comparison to vehicle, in CYP-injected rats, effects of compound I-B (500 mg/kg, p.o.) led to:
A significant decrease in nociceptive scores at +2, +4 h and +6 h.
A significant increase in nociceptive thresholds for 6 hours following CYP injection.

In comparison to vehicle, in CYP-injected rats, effects of compound I-C (500 mg/kg, p.o.) led to:
A significant decrease in nociceptive scores at +2, +4 h and +6 h,
A significant increase in nociceptive thresholds for 6 hours following CYP injection.

### III. Conclusion

A single intraperitoneal injection of CYP (150 mg/kg) induced visceral pain at 2-, 4- and 6-hours post-injection, thus validating the model.

Single oral treatment of NMN (500 mg/kg) alleviated CYP-induced visceral pain at both evaluated time points (+2 h and +4 h) with a higher level of significance at +4 h.

Pro-drug A (500 mg/kg, p.o.) reduced CYP-induced visceral pain at both evaluated time points (+2 h and +4 h) with significance at +4 h.

In CYP-injected rats, oral treatment with Pro-drug B (compound I-A) (500 mg/kg, p.o.) resulted in significant anti-nociceptive activity at +4 h.

In CYP-injected rats, oral treatment with compounds I-B and I-C (500 mg/kg, p.o.) displayed significant anti-nociceptive activity at the three evaluated time points, +2 h, +4 h and +6 h.

### Example 3: Evaluation of Compounds of the Invention in a Model of Doxorubicin-Induced Cardiotoxicity

The aim of the present study was to evaluate, the effects of i.p administration of Nicotinamide Mononucleotide (NMN), compound I-B and compound I-C at 180 mg/kg in the progression of a cardiotoxicity induced by doxorubicin.

### I. Materials and Methods

### Material

### Animals:

76 male mice, 8-week-old at the arrival were obtained from Janvier Labs, Le Genest St Isle, 53941 St Berthevin, France. Each animal was identified with electronic chip. Each cage was numbered. Based on the animal number/cage and number of cages, the animals were assigned of unique number with the name of group and mice number.

The matching cards that were used to identify cages where experimental animals were housed contained the following information: the name of the experiment, the number of the experiment and the cage number.

### Methods

1. Preparation of Formulation:

The powder of NMN, compounds I-B and I-C (180 mg/kg) were dissolved in vehicle (the solution is used at room temperature for maximum 1 day). A fresh sample for each administration was prepared every day except the week-end (the solution is prepared on Saturday and is used on Saturday and Sunday).

2. Doxororubicin-induced cardiotoxicity

Cardiotoxicity was induced by a single intraperitoneal injection of doxorubicin (DOX) at 20 mg/kg. Doxorubicin was prepared at 2 mg/mL and volume of administration was 10 mL/Kg.

Mortality rate was followed-up all along the experimental phase.

3. Experimental Groups

Group description:

Group 1: Vehicle (i.p.)

Group 2: Doxorubicin (20 mg/kg)

Group 3: Doxorubicin (20 mg/kg)+test compound 180 mg/kg (NMN)

Group 4: Doxorubicin (20 mg/kg)+test compound 180 mg/kg (compound I-B)

Group 5: Doxorubicin (20 mg/kg)+test compound 180 mg/kg (compound I-C)

Group repartition:

Each group involved 14-24 mice.

As set forth in the regulations for Non-clinical Laboratory Studies, test and control animal groups were maintained under identical conditions. The intended duration of study was 11 days.

4. Induction with Doxorubicin

At D0, mice were injected with DOX (20 mg/kg) by intraperitoneal route.

5. Treatment

The treatment with NMN, compounds I-B and I-C was initiated from 5 days before DOX injection, once per day, from D-5 to DO.

Mice were i.p treated with NMN, compounds I-B and I-C 30 min before DOX injection.

Mice were i.p treated with NMN, compounds I-B and I-C for the duration of the experiment (D0 to D5) once per day. Last injection occurred 24 hours before sacrifice.

6. Body Weight, Survival Rate and Clinical Examination

The bodyweight was assessed at inclusion and at D5.

The survival rate was recorded every day until the end of the experiment (D5).

7. Blood Collection

Retro-orbital blood collection was performed at the inclusion and at 1 and 5 days after the infection with DOX to assess biomarker (LDH and creatinine).

8. Organs Collection

At D5, heart, and tibia were collected.

9. Assessment of Cardiac Function by Echocardiography

Echocardiography (ECG) was performed 5 days after doxorubicin injection in anesthetized (isoflurane 1.5-2%) animals with non-invasive two-dimensional echocardiography (VF16-5 probe, Siemens, Acuson NX3 Elite). After removing hairs on the chest, numeric images of the heart were obtained in both parasternal long-axis and short axis views.

The following cardiac function were assessed during ECG:
Left ventricle (LV) end-systolic and end-diastolic inner diameter;
LV end-systolic and end-diastolic volume,
Fractionnal shortening;
Ejection fraction;
Heart Rate; and
Anterior and posterior wall thickness in Diastole and in systole.

### II. Results and Discussion

1. Survival Rate

FIG. 9 shows the percentage of survival of mice induced or not with DOX (20 mg/kg), 5 days after doxorubicin injection.

DOX mice were treated with NMN, compounds I-B and I-C (180 mg/kg) or vehicle.

As shown in FIG. 9, almost 50% of doxorubicin mice treated with vehicle died before the end of experimental protocol.

Treatment with NMN tended to improve the survival rate (78% of survival) without reaching statistical significance. However, treatment with compounds I-B or I-C significantly improved survival rate (98% and 100% of survival respectively) compared to untreated groups (50% of survival).

2. Body Weight

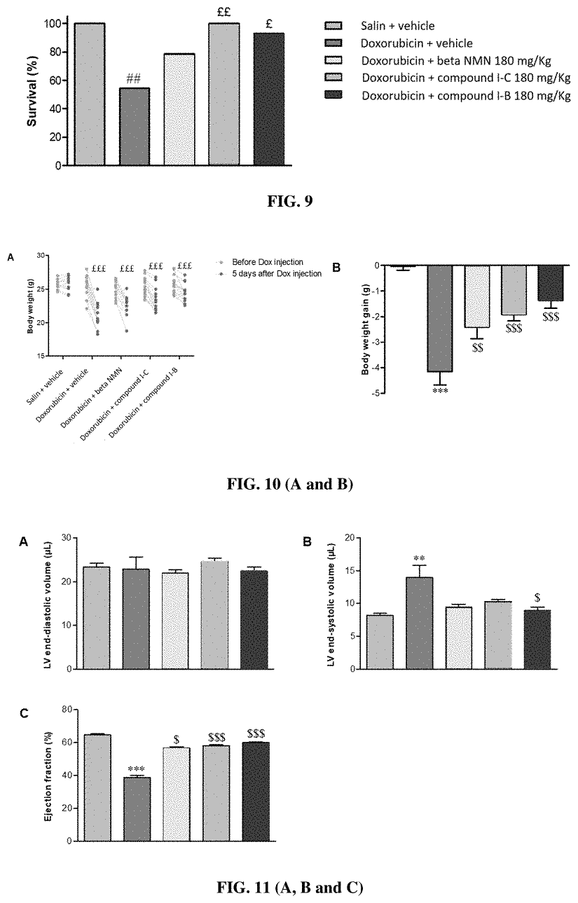

FIG. 10A shows the body weight evolution of mice treated with NMN, compounds I-B and I-C (180 mg/kg) or vehicle, before (light gray symbol) and 5 days after saline solution or DOX (20 mg/kg) injection (dark gray symbol).

FIG. 10B shows the body weight gain calculated as follow: bodyweight at the day of sacrifice minus bodyweight before injection.

Surviving vehicle-treated mice showed major signs of suffering associated with a strong decrease in body weight (−4.2±0.5 g). The body weight loss observed after doxorubicin administration was significantly decreased by NMN, compounds I-B and I-C (p<0.01, p<0.001, p<0.001 respectively).

3. Cardiac Function

3.1. Left Ventricle End Diastolic/Systolic Volumes and Ejection Fraction

FIG. 11 shows Left ventricle (LV) end diastolic (FIG. 11A), end systolic volumes (FIG. 11B) and ejection fraction (FIG. 11C) 5 days after saline solution or DOX (20 mg/kg) injection, with and without treatment with NMN, compound I-B and compound I-C.

As shown in FIG. 11B, doxorubicin induced an significant increase in end-systolic LV (left ventricular) volume without difference in end-diastolic (FIG. 11A) when compared to control group leading to a large decrease in ejection fraction (38.9±1.3% in doxorubicin vehicle group vs 64.8±0.6% in control mice) (FIG. 11C).

Compared to untreated DOX animals, NMN, compounds I-B and I-C reduced end-systolic LV (FIG. 11B) volume when compared to doxorubicin vehicle group, with statistical significance observed for compound I-B (p<0.05).

Compared to DOX-induced animals that received vehicle, ejection fraction was significantly improved after treatment with NMN. compounds I-B and I-C (56.9±0.6% in doxorubicin mice treated with NMN (p<0.05). 58.2±0.5% in doxorubicin mice treated with compound I-C (p<0.001) and 60.0±0.6% in doxorubicin mice treated with compound I-B (p<0.001)) (FIG. 11C).

3.2. Left Ventricle End Diastolic/Systolic Diameters, Fractional Shortening and Heart Rate

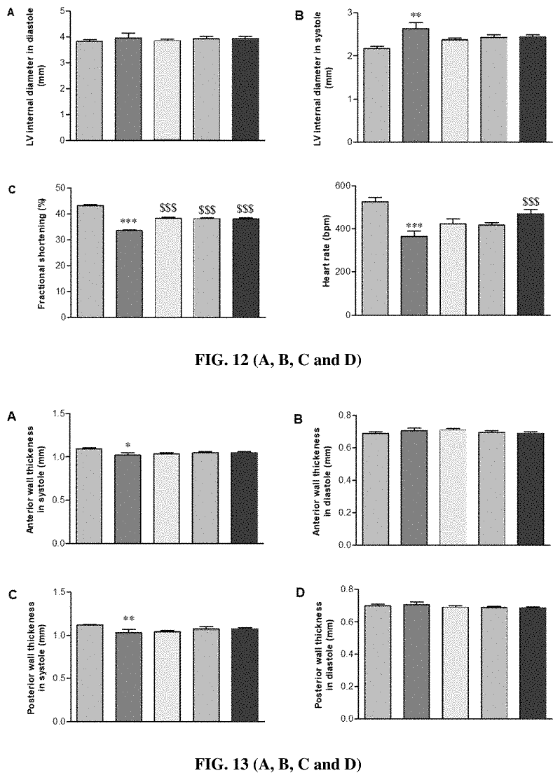

FIG. 12 shows LV end-diastolic and end-systolic diameters (FIGS. 12A and 12B respectively), fractional shortening (FIG. 12C) and heart rate (FIG. 12D) 5 days after saline solution or DOX (20 mg/kg) injection.

As shown, in doxorubicin-treated mice, LV internal diameter was significantly increased in systole (FIG. 12B) without significative difference in diastole (FIG. 12A) resulting in a decrease in fractional shortening (33.5±0.4% vs 43.2±0.5% in control mice) (FIG. 12C). Treatment with NMN. compounds I-B and I-C significantly improved fractional shortening to around 38% (p<0.001 for the three groups).

Moreover. doxorubicin significantly reduced the heart rate (FIG. 12D) when compared to control mice (365.1±23.9 bpm vs 525.6±19.8 bpm respectively). Treatments with NMN. compound I-B and compound I-C resulted in increased heart rates. with compound I-B significantly improving this parameter (470.1±18.8 bpm (p<0.001)).

3.3. Left Ventricle Anterior and Posterior Wall Thickness in Systole and in Diastole

FIG. 13 shows LV anterior wall thickness in systole and in diastole (FIGS. 13A and 13B respectively) and posterior wall thickness in systole and in diastole (FIGS. 13C and 13D respectively) 5 days after saline solution or DOX (20 mg/kg) injection.

Doxorubicin significantly decreased anterior and posterior wall thickness in systole but not in diastole, and any of treatments had significant effect.

Treatments with compound NMN, compounds I-B and I-C (180 mg/kg) in DOX mice resulted in non-significant increases of anterior and posterior wall thickness in systole.

4. Heart Weight

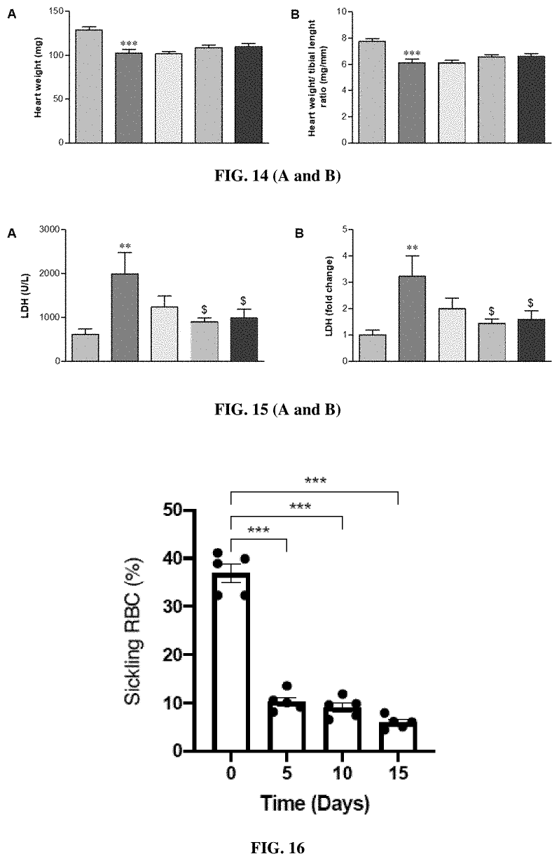

FIG. 14 shows Heart weight (FIG. 14A) and heart weight normalized to tibial length (FIG. 14B) 5 days after saline solution or DOX (20 mg/kg) injection.

DOX mice were treated with compound NMN, compounds I-B and I-C (180 mg/kg) or vehicle.

As shown in FIGS. 14A and 14B, doxorubicin significantly decreased heart weight when compared to control mice (102.3±4.6 mg vs 128.9±3.3 mg respectively). Treatments with compounds I-B and I-C tended to increase heart weight without reaching significance vs DOX vehicle mice. Similar results were obtained when heart weight was normalized to tibia length.

5. Biomarker Assessment

FIG. 15 shows LDH concentrations (U/L, FIG. 15A) and LDH (fold change, FIG. 15B) in the plasma of mice 5 days after saline solution or DOX (20 mg/kg) injection.

DOX mice were treated with compound NMN, compounds I-B and I-C (180 mg/kg) or vehicle.

Plasmatic LDH (lactate deshydrogenase) were measured 5 days after doxorubicin injection. As shown in FIGS. 15A and 15B, doxorubicin induced a 3-fold increase in LDH release compared to control group. Treatment with NMN decreased LDH release by more than 35% without reaching statistical significance. However, treatment with both compounds I-B and I-C resulted in a significant reduction in LDH levels by 50-55% (p<0.05).

III. Conclusion

Altogether, results showed that doxorubicin induced cardiac dysfunction characterized by impaired cardiac contractility and cardiac filling, as well as cell cardiac damage. Doxorubicin also led to high mortality, and a strong body weight loss.

NMN, compounds I-B and I-C treatments significantly improved survival rate, body weight loss and prevented cardiac function degradation as shown by the effects of treatments on ejection fraction, fractional shortening and heart rate.

### Example 4: Evaluation of Compounds of the Invention on Sickle Cell Disease Experimental Models

The aim of the present study was to evaluate, the effects of i.p. administration of Nicotinamide Mononucleotide (NMN), compound I-B and compound I-C at 185 mg/kg as modulator of red blood cell sickling and their potential role in therapy for sickle cell disease on a mouse model of SCD.

I. Materials and Methods

Material

Animals:

Townes S/S mice on a 129/B6 mixed genetic background.

Methods

1. Preparation of Formulation:

The powder of NMN, compounds I-B and I-C (185 mg/kg) were dissolved in vehicle (the solution was used at room temperature for maximum 1 day). A fresh sample for each administration was prepared every day except the week-end (the solution is prepared on Saturday and is used on Saturday and Sunday).

2. Sickle Red Blood Cell

In Townes S/S mice, mouse alpha- and beta-globin gene loci are deleted and replaced by human alpha- and beta-globins. When carrying two copies of the beta S allele, mice develop a human sickle cell disease phenotype with sickle-shaped red blood cells seen in blood smears.

3. Experimental Groups

Group description:

Group I: Vehicle (i.p.)

Group II: NMN 185 mg/kg

Group III: compound I-B 185 mg/kg

Group IV compound I-C 185 mg/kg

4. Treatment

Mice were i.p treated with NMN, compounds I-B and I-C during all the experiment (D0 to D15) once per day. Last injection occurred 24 hours before sacrifice.

5. Blood Collection

Retro-orbital blood collection was performed at the inclusion D0 and at D5, D10 and D15 through facial vein bleeding.

6. Ex Vivo

Red blood cells collected were submitted to hypoxia (1% O₂) for 30 minutes to induce sickling. Percentage of sickle cells was then assessed.

I. Results and Discussion

1. RBC Sickling Under Hypoxia Ex Vivo

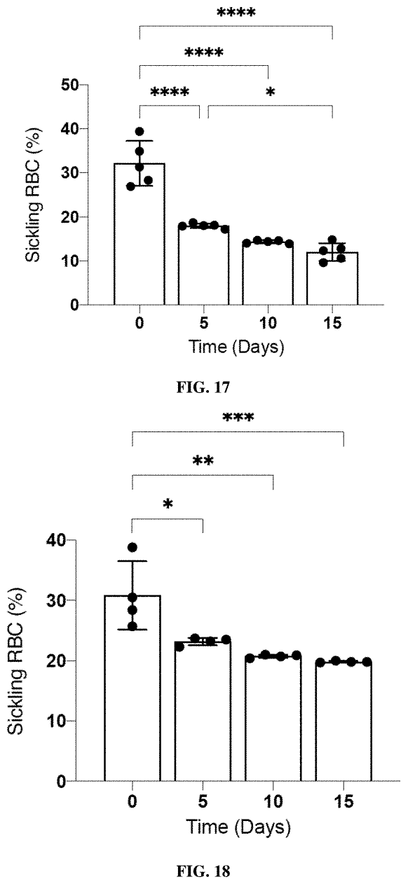

FIGS. 16, 17 and 18 show the ability of NMN (FIG. 16), compounds I-B (FIG. 17) and I-C (FIG. 18) to prevent sickling of SS RBCs at a 1% O₂.

SS RBCs from treated mice were collected at D0, D5, D10 and D15 and were submitted to hypoxia for 30 minutes in a hypoxic chamber (1% O₂). Percentage of sickling RBCs was then assessed for each time point with compounds NMN, I-B and I-C.

The results showed that treatment with:
NMN (185 mg/kg, i.p.) led to a significant (p<0.001) decrease of the percentage of sickling cells from 40% at D0 to less than 10% after 15 days treatment of mice (FIG. 16);
Compound I-B (185 mg/kg, i.p.) led to a significant decrease (p<0.0001) of the percentage of sickling cells from 32% at D0 to less than 15% after 15 days treatment of mice (FIG. 17).
Compound I-C (185 mg/kg, i.p.) led to a significant decrease (p<0.001) of the percentage of sickling cells from 31% at D0 to 20% after 15 days treatment of mice (FIG. 18).

III. Conclusion

These results indicate that NMN, compounds I-B and I-C prevented SS RBCs sickling under hypoxic conditions.

1.-17. (canceled)

18. A compound of formula I 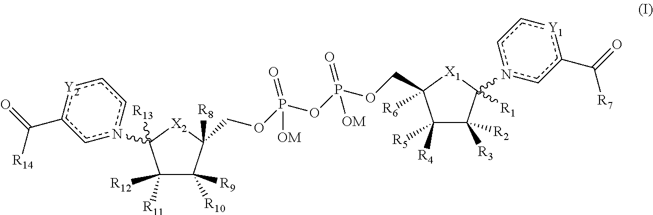
or pharmaceutically acceptable salts and/or solvates thereof, for use as a medicament, wherein:
X₁ and X₂ are independently selected from O, CH₂, S, Se, CHF, CF₂ and C═CH₂;
R₁ and R₁₃ are independently selected from H, azido, cyano, C1-C8 alkyl, C1-C8 thio-alkyl, C1-C8 heteroalkyl and OR, wherein R is selected from H and C1-C8 alkyl;
R₂, R₃, R₄, R₅, R₉, R₁₀, R₁₁, R₁₂ are independently selected from H, halogen, azido, cyano, hydroxyl, C1-C12 alkyl, C1-C12 thio-alkyl, C1-C12 heteroalkyl, C1-C12 haloalkyl and OR; wherein R is selected from H, C1-C12 alkyl, C(O)(C1-C12)alkyl, C(O)NH(C1-C12)alkyl, C(O)O(C1-C12)alkyl, C(O)aryl, C(O)(C1-C12)alkyl aryl, C(O)NH(C1-C12)alkyl aryl, C(O)O(C1-C12)alkyl aryl or C(O)CHR_{AA}NH₂, wherein R_{AA} is a side chain selected from a proteinogenic amino acid;
R₆ and R₈ are independently selected from H, azido, cyano, C1-C8 alkyl and OR; wherein R is selected from H and C1-C8 alkyl;
R₇ and R₁₄ are independently selected from H, OR, NHR, NRR′, NH—NHR, SH, CN, N₃ and halogen; wherein R and R′ are each independently selected from H, C1-C8 alkyl, (C1-C8)alkyl aryl;
Y₁ and Y₂ are independently selected from CH, CH₂, C(CH₃)₂ or CCH₃;
M is selected from H or a suitable counterion; [symbol]
represents a single or a double bound depending on Y₁ and Y₂; and [symbol]
represents the alpha or beta anomer depending on the position of R₁ and R₁₃.

19. The compound for use according to claim 18, wherein X₁ and X₂ each independently represents an oxygen.

20. The compound for use according to claim 18, wherein R₁ and/or R₁₃ each independently represents a hydrogen.

21. The compound for use according to claim 18, wherein R₆ and/or R₈ each independently represents a hydrogen.

22. The compound for use according to claim 18, wherein R₃, R₄, R₁₀, R₁₁ are identical and represent each a hydrogen.

23. The compound for use according to claim 18, wherein R₂, R₅, R₉ and R₁₂ are identical and represent each a hydroxyl.

24. The compound for use according to claim 18, wherein Y₁ and Y₂ each independently represents a CH.

25. The compound for use according to claim 18, wherein Y₁ and Y₂ each independently represents a CH₂.

26. The compound for use according to claim 18, selected from compounds of formula I-A to I-F:

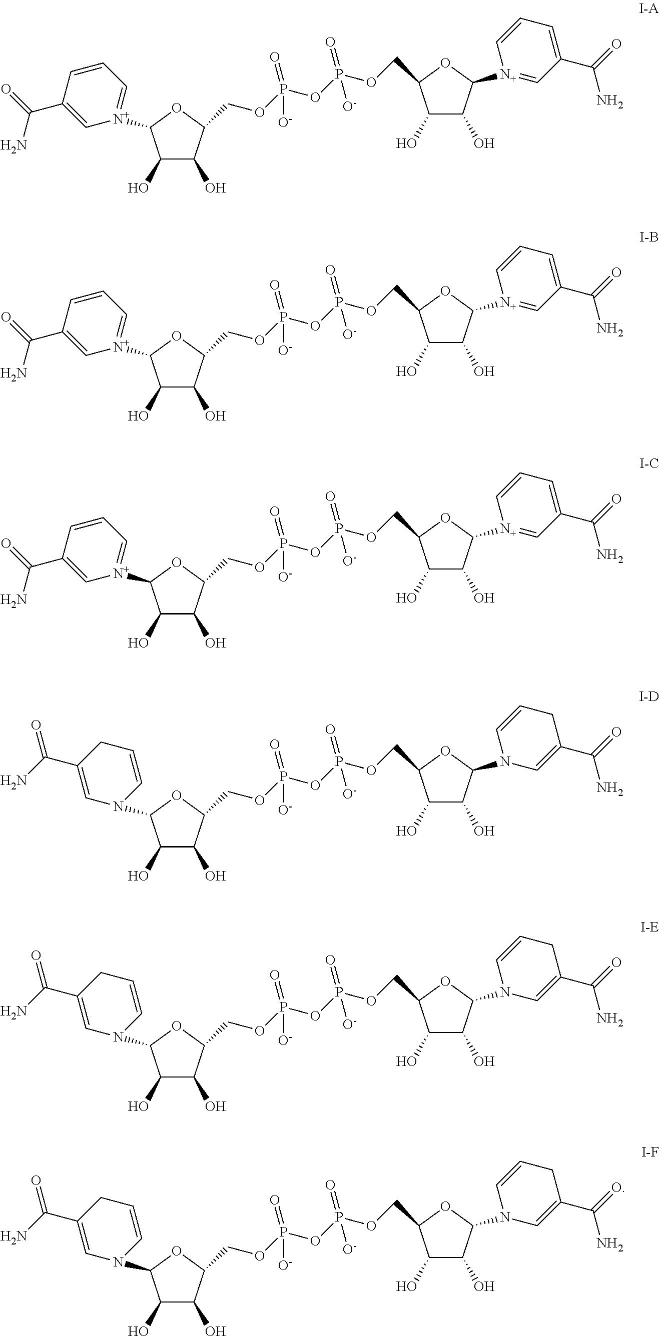

27. The compound for use according to claim 18, The compound for use according to claim 18, wherein the compound is of formula I-A, I-B or I-C.

28. A compound of formula I′ 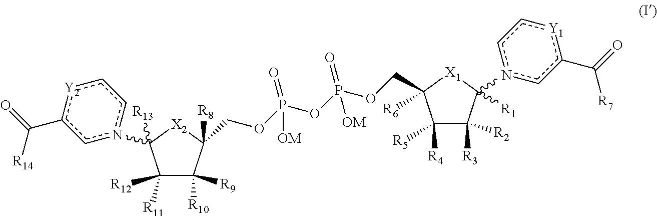
or pharmaceutically acceptable salts and/or solvates thereof, wherein:
X₁ and X₂ are independently selected from O, CH₂, S, Se, CHF, CF₂ and C═CH₂;
R₁ and R₁₃ are independently selected from H, azido, cyano, C1-C8 alkyl, C1-C8 thio-alkyl, C1-C8 heteroalkyl and OR, wherein R is selected from H and C1-C8 alkyl;
R₂, R₃, R₄, R₅, R₉, R₁₀, R₁₁, R₁₂ are independently selected from H, halogen, azido, cyano, hydroxyl, C1-C12 alkyl, C1-C12 thio-alkyl, C1-C12 heteroalkyl, C1-C12 haloalkyl and OR; wherein R is selected from H, C1-C12 alkyl, C(O)(C1-C12)alkyl, C(O)NH(C1-C12)alkyl, C(O)O(C1-C12)alkyl, C(O)aryl, C(O)(C1-C12)alkyl aryl, C(O)NH(C1-C12)alkyl aryl, C(O)O(C1-C12)alkyl aryl or C(O)CHR_{AA}NH₂, wherein R_{AA} is a side chain selected from a proteinogenic amino acid;
R₆ and R₈ are independently selected from H, azido, cyano, C1-C8 alkyl and OR; wherein R is selected from H and C1-C8 alkyl;
R₇ and R₁₄ are independently selected from H, OR, NHR, NRR′, NH—NHR, SH, CN, N₃ and halogen; wherein R and R′ are each independently selected from H, C1-C8 alkyl, (C1-C8)alkyl aryl;
Y₁ and Y₂ are independently selected from CH, CH₂, C(CH₃)₂ or CCH₃;
M is selected from H or a suitable counterion; [symbol]
represents a single or a double bound depending on Y₁ and Y₂; and [symbol]
represents the alpha or beta anomer depending on the position of R₁ and R₁₃,
with the proviso that when; X₁ and X₂ are oxygen; R₁, R₃, R₄, R₆, R₅, R₁₀, R₁₁, and R₁₃ are hydrogen; R₂, R₅, R₉ and R₁₂ are hydroxyl; R₇ and R₁₄ are NH₂; and Y₁ and Y₂ are independently selected from CH or CH₂,
then at least one of [symbol] represent the alpha anomer.

29. A pharmaceutical composition comprising at least one compound for use according to claim 18, and at least one pharmaceutically acceptable carrier.

30. A method of treatment of pain, antineoplastic-induced cardiotoxicity or sickle cell disease in a subject, comprising administering to a subject in need thereof an effective amount of the compound for use according to claim 18.

31. A food composition comprising at least one compound for use according to claim 18, and at least one acceptable carrier and/or diluent.

32. A cosmetic composition comprising at least one compound for use according to claim 18, and at least one acceptable carrier and/or diluent.

33. A method for preparing a compound of formula I as defined in claim 18 comprising the following steps:
1) mono-phosphorylation of a compound of formula X, 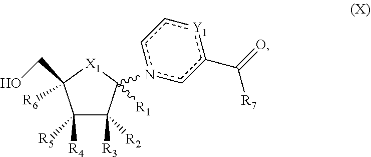
wherein:
X₁, R₁, R₂, R₃, R₄, R₅, R₆, R₇, Y₁, [symbol] and [symbol] are as defined above, to give compound of formula XI, 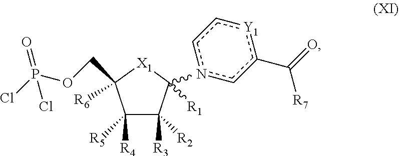
wherein:
X₁, R₁, R₂, R₃, R₄, R₅, R₆, R₇, Y₁, [symbol] and [symbol] are as defined above;
2) hydrolysis of compound of formula XI obtained in step 1), to give compound of formula XII 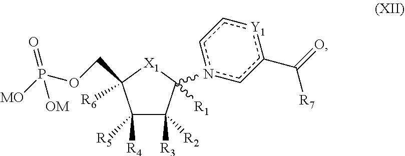
wherein:
X₁, R₁, R₂, R₃, R₄, R₅, R₆, R₇, Y₁, [symbol] and [symbol] are as defined above;
3) reacting compound of formula XII obtained in step 2) with compound of formula XIII, 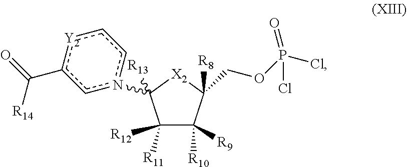
obtained as described in step 1) and wherein:
X₂, R₈, R₉, R₁₀, R₁₁, R₁₂, R₁₃, R₁₄, Y₂, [symbol] and [symbol] are as defined above, to give compound of formula I.

34. The method according to claim 33, further comprising a step of reducing the compound of formula I or formula I′ obtained in step 3), to give the compound of formula I, wherein Y₁ and Y₂ each independently represents a CH₂.
